SEQUENCE LISTING (64 sequences)

SEQ ID NO: 1 - protein, 550 aa, synthetic construct
  REGION 1..550: note = synthetically generated polypeptide
MAPVAVWAALAVGLELWAAALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGIEGPTLRQWLAARAGGGGGGGGIEGPTLRQWLAARAGGGGGGGGIEGPTLRQWLAARA

SEQ ID NO: 2 - protein, 530 aa, synthetic construct
  REGION 1..530: note = synthetically generated polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGIEGPTLRQWLAARAGGGGGGGGIEGPTLRQWLAARAGGGGGGGGIEGPTLRQWLAARA

SEQ ID NO: 3 - protein, 20 aa, synthetic construct
  REGION 1..20: note = synthetically generated polypeptide
MAPVAVWAALAVGLELWAAA

SEQ ID NO: 4 - protein, 235 aa, synthetic construct
  REGION 1..235: note = synthetically generated polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 5 - protein, 232 aa, synthetic construct
  REGION 1..232: note = synthetically generated polypeptide
EPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 63 aa, synthetic construct
  REGION 1..63: note = synthetically generated polypeptide
GGGGGIEGPTLRQWLAARAGGGGGGGGIEGPTLRQWLAARAGGGGGGGGIEGPTLRQWLAARA

SEQ ID NO: 7 - protein, 14 aa, synthetic construct
  REGION 1..14: note = synthetically generated polypeptide
IEGPTLRQWLAARA

SEQ ID NO: 8 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 9 - protein, 41 aa, synthetic construct
  REGION 1..41: note = synthetically generated polypeptide
GGGGGIEGPTLRQWLAARAGGGGGGGGIEGPTLRQWLAARA

SEQ ID NO: 10 - protein, 508 aa, synthetic construct
  REGION 1..508: note = synthetically generated polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGIEGPTLRQWLAARAGGGGGGGGIEGPTLRQWLAARA

SEQ ID NO: 11 - protein, 4 aa, synthetic construct
GGGG

SEQ ID NO: 12 - protein, 5 aa, synthetic construct
GGGGG

SEQ ID NO: 13 - protein, 6 aa, synthetic construct
GGGGGG

SEQ ID NO: 14 - protein, 8 aa, synthetic construct
GGGGGGGG

SEQ ID NO: 15 - protein, 4 aa, synthetic construct
GGGA

SEQ ID NO: 16 - protein, 4 aa, synthetic construct
GGGG

SEQ ID NO: 17 - protein, 5 aa, synthetic construct
GGGAG

SEQ ID NO: 18 - protein, 6 aa, synthetic construct
GGGAGG

SEQ ID NO: 19 - protein, 7 aa, synthetic construct
GGGAGGG

SEQ ID NO: 20 - protein, 4 aa, synthetic construct
GGGS

SEQ ID NO: 21 - protein, 5 aa, synthetic construct
GGGGA

SEQ ID NO: 22 - protein, 5 aa, synthetic construct
GGGGS

SEQ ID NO: 23 - protein, 4 aa, synthetic construct
GGAG

SEQ ID NO: 24 - protein, 4 aa, synthetic construct
GGSG

SEQ ID NO: 25 - protein, 4 aa, synthetic construct
AGGG

SEQ ID NO: 26 - protein, 4 aa, synthetic construct
SGGG

SEQ ID NO: 27 - protein, 4 aa, synthetic construct
GAGA

SEQ ID NO: 28 - protein, 4 aa, synthetic construct
GSGS

SEQ ID NO: 29 - protein, 6 aa, synthetic construct
GAGAGA

SEQ ID NO: 30 - protein, 6 aa, synthetic construct
GSGSGS

SEQ ID NO: 31 - protein, 8 aa, synthetic construct
GAGAGAGA

SEQ ID NO: 32 - protein, 8 aa, synthetic construct
GSGSGSGS

SEQ ID NO: 33 - protein, 10 aa, synthetic construct
GAGAGAGAGA

SEQ ID NO: 34 - protein, 10 aa, synthetic construct
GSGSGSGSGS

SEQ ID NO: 35 - protein, 12 aa, synthetic construct
GAGAGAGAGAGA

SEQ ID NO: 36 - protein, 12 aa, synthetic construct
GSGSGSGSGSGS

SEQ ID NO: 37 - protein, 6 aa, synthetic construct
GGAGGA

SEQ ID NO: 38 - protein, 6 aa, synthetic construct
GGSGGS

SEQ ID NO: 39 - protein, 9 aa, synthetic construct
GGAGGAGGA

SEQ ID NO: 40 - protein, 9 aa, synthetic construct
GGSGGSGGS

SEQ ID NO: 41 - protein, 12 aa, synthetic construct
GGAGGAGGAGGA

SEQ ID NO: 42 - protein, 12 aa, synthetic construct
GGSGGSGGSGGS

SEQ ID NO: 43 - protein, 8 aa, synthetic construct
GGAGGGAG

SEQ ID NO: 44 - protein, 8 aa, synthetic construct
GGSGGGSG

SEQ ID NO: 45 - protein, 12 aa, synthetic construct
GGAGGGAGGGAG

SEQ ID NO: 46 - protein, 12 aa, synthetic construct
GGSGGGSGGGSG

SEQ ID NO: 47 - protein, 15 aa, synthetic construct
GGGGAGGGGAGGGGA

SEQ ID NO: 48 - protein, 15 aa, synthetic construct
GGGGSGGGGSGGGGS

SEQ ID NO: 49 - protein, 4 aa, synthetic construct
AAAL

SEQ ID NO: 50 - protein, 4 aa, synthetic construct
AAAK

SEQ ID NO: 51 - protein, 4 aa, synthetic construct
AAAR

SEQ ID NO: 52 - protein, 14 aa, synthetic construct
EGKSSGSGSESKST

SEQ ID NO: 53 - protein, 12 aa, synthetic construct
GSAGSAAGSGEF

SEQ ID NO: 54 - protein, 12 aa, synthetic construct
AEAAAKEAAAKA

SEQ ID NO: 55 - protein, 18 aa, synthetic construct
KESGSVSSEQLAQFRSLD

SEQ ID NO: 56 - protein, 10 aa, synthetic construct
GENLYFQSGG

SEQ ID NO: 57 - protein, 8 aa, synthetic construct
SACYCELS

SEQ ID NO: 58 - protein, 5 aa, synthetic construct
RSIAT

SEQ ID NO: 59 - protein, 17 aa, synthetic construct
RPACKIPNDLKQKVMNH

SEQ ID NO: 60 - protein, 36 aa, synthetic construct
GGSAGGSGSGSSGGSSGASGTGTAGGTGSGSGTGSG

SEQ ID NO: 61 - protein, 17 aa, synthetic construct
AAANSSIDLISVPVDSR

SEQ ID NO: 62 - protein, 36 aa, synthetic construct
GGSGGGSEGGGSEGGGSEGGGSEGGGSEGGGSGGGS

SEQ ID NO: 63 - protein, 5 aa, synthetic construct
EAAAK

SEQ ID NO: 64 - protein, 5 aa, synthetic construct
PAPAP

(19) United States
(12) Patent Application Publication
(10) Pub. No.: US 2024/0391977 A1
(43) Pub. Date: Nov. 28, 2024

(54) TPO MIMETIC FUSION PROTEINS AND METHODS OF USE

(71) Applicant: SICHUAN CLOVER BIOPHARMACEUTICALS, INC., Chengdu, Sichuan (CN)

(72) Inventor: Peng LIANG, Chengdu, Sichuan (CN)

(21) Appl. No.: 18/694,818

(22) PCT Filed: Sep. 23, 2022
(86) PCT No.: PCT/CN2022/120948

§ 371 Date: Mar. 22, 2024

(30) Foreign Application Priority Data
Sep. 24, 2021 (WO) PCT/CN2021/120388

(51) Int. Cl. C07K 14/715; A61K 38/00; C07K 14/52

(52) U.S. Cl. CPC C07K 14/7151; C07K 14/524; A61K 38/00; C07K 2319/30

(57) ABSTRACT

The recombinant peptides and proteins having thrombopoietin (TPO) activities, e.g., thrombopoietin mimetic peptides that are TPO receptor (TPOR) agonists. The recombinant peptides or proteins include a recombinant human p75 TNF receptor and TPO mimetic peptides linked by an Fc fragment of IgG1. The recombinant peptides form a homodimer by inter-chain disulfide bond in the Fc region and include six TPOR binding and/or activating domain in the C-terminus. The recombinant peptides or proteins provided herein are useful for increasing platelet count, e.g., for treating thrombocytopenia. The methods for producing the recombinant peptides and proteins, formulations, and therapeutic methods.

The content of the following submission on ASCII text file is incorporated herein by reference in its entirety: a computer readable form (CRF) of the Sequence Listing (file name: 165762001240SEQLIST.TXT, date recorded: Sep. 24, 2021, size: 23 KB).

### FIELD

The present disclosure relates in some aspects to fusion peptides and proteins that have thrombopoietin (TPO) activities, e.g., thrombopoietin receptor agonists and/or thrombopoietin mimetic peptides, and tumor necrosis factor α (TNF-α) receptor activities, and methods of using such recombinant peptides and proteins, for instance, for increasing production of platelets and/or its precursors, and for treating thrombocytopenia.

### BACKGROUND

Thrombocytopenia has many causes, including autoimmune disorder (e.g. chronic immune (idiopathic) thrombocytopenic purpura (ITP)), chemotherapy (e.g. chemotherapy-induced thrombocytopenia (CIT)), immune-oncology therapy, and liver inflammations or damages. Severe thrombocytopenia could be corrected by platelet infusion. However, repeated infusion of platelet could cause serious side effect and even exacerbate thrombocytopenia. Additionally, the supply of platelet is limited. On the other hand, currently available drugs for thrombocytopenia are limited by safety risks or need very frequent administration. Therefore, there are still unmet needs for thrombocytopenia treatments. Tumor patients need safer, more efficient, more economical, and more convenient thrombocytopenia treatments to improve treatment experience and quality of life.

### SUMMARY

In some embodiments, disclosed herein is a polypeptide comprising a tumor necrosis factor (TNF) binding and/or inhibiting moiety and a thrombopoietin receptor (TPOR) binding and/or activating moiety. In some embodiments, the TNF binding and/or inhibiting moiety can be a TNF receptor (TNFR) moiety that binds to TNF-α or an anti-TNF-α antibody or antigen binding fragment thereof, and wherein the TPOR binding and/or activating moiety can comprise a TPOR binding and/or activating domain.

In any of the embodiments herein, the TNF binding and/or inhibiting moiety can comprise a human TNFR2 (p75) or a functional fragment or variant thereof or a human TNFR1 (p55) or a functional fragment or variant thereof. In any of the embodiments herein, the TNF binding and/or inhibiting moiety (e.g., TNFR moiety) can be an extracellular portion of human TNFR2. In any of the embodiments herein, the TNF binding and/or inhibiting moiety comprises infliximab (e.g., Remicade®) or a biosimilar, bioequivalent, or biobetter thereof, golimumab (e.g., Simponi®) or a biosimilar, bioequivalent, or biobetter thereof, adalimumab (e.g., Humira®) or a biosimilar, bioequivalent, or biobetter thereof, certolizumab pegol (e.g., Cimzia®) or a biosimilar, bioequivalent, or biobetter thereof, or an antigen binding fragment thereof.

In any of the embodiments herein, the TPOR binding and/or activating moiety can comprise one, two, three, or more TPOR binding and/or activating domains. In any of the embodiments herein, the polypeptide can comprise one or more spacers between two TPOR binding and/or activating domains. In any of the embodiments herein, the TPOR binding and/or activating domain can be derived from human thrombopoietin (TPO). In any of the embodiments herein, the TPOR binding and/or activating domain can comprise a human thrombopoietin mimetic (TPM) peptide.

In any of the embodiments herein, the TNF binding and/or inhibiting moiety and the TPOR binding and/or activating moiety can be linked by an immunoglobulin Fc moiety. In any of the embodiments herein, the immunoglobulin Fc moiety can comprise an IgG, IgM, IgD, IgA, or IgE Fc region or a fragment or variant thereof. In any of the embodiments herein, the immunoglobulin Fc moiety can comprise a human IgG1, IgG2, IgG3, or IgG4 Fc region or a fragment or variant thereof.

In any of the embodiments herein, the TNF binding and/or inhibiting moiety can be fused to the immunoglobulin Fc moiety which can in turn be fused to the TPOR binding and/or activating moiety. In any of the embodiments herein, the C terminus of the TNF binding and/or inhibiting moiety can be fused to the N terminus of the immunoglobulin Fc moiety, and the C terminus of the immunoglobulin Fc moiety can be fused to the N terminus of the TPOR binding and/or activating moiety.

In some embodiments, disclosed herein is a polypeptide comprising a sequence of the formula: TNFR-Fc-(S₁)ₘ-TPORBD₁-(S₂)ₙ-TPORBD₂-(S₃)ₚ-TPORBD₃, wherein TNFR is a tumor necrosis factor receptor or a fragment or variant thereof; Fc is an immunoglobulin Fc region or a fragment or variant thereof; TPORBD₁, TPORBD₂, and TPORBD₃ are the same or different thrombopoietin receptor (TPOR) binding and/or activating domains; S₁, S₂, and S₃ are the same or different spacers; and m, n, and p are 0 or greater and are integers independent of one another.

In any of the embodiments herein, the TNFR can comprise a sequence having at least 90%, at least 91%, at least 92%, at least 93%, at least 94%, at least 95%, at least 96%, at least 97%, at least 98%, at least 99%, or 100% sequence identity with SEQ ID NO: 4.

In any of the embodiments herein, the Fc can comprise a sequence having at least 90%, at least 91%, at least 92%, at least 93%, at least 94%, at least 95%, at least 96%, at least 97%, at least 98%, at least 99%, or 100% sequence identity with SEQ ID NO: 5. In any of the embodiments herein, the Fc can comprise at least an N-glycosylation site mutation compared to a wild type human Fc, optionally wherein the N-glycosylation site mutation is at N314 according to Kabat numbering (N297 according to EU numbering).

In any of the embodiments herein, m, n, and p can be independently selected from 1 to 10. In any of the embodiments herein, each of S₁, S₂, and S₃ can comprise a peptide linker. In any of the embodiments herein, each of S₁, S₂, and S₃ can comprise a plurality of glycine, alanine, serine, and/or leucine residues. In any of the embodiments herein, each of S₁, S₂, and S₃ can comprise between about five and about eight consecutive glycine residues. In any of the embodiments herein, one or more of S₁, S₂, and S₃ can comprise at least five consecutive glycine residues. In any of the embodiments herein, each of TPORBD₁, TPORBD₂, and TPORBD₃ can comprise a sequence having at least 80%, at least 85%, at least 90%, at least 95%, at least 99%, or 100% sequence identity with SEQ ID NO: 7. In any of the embodiments herein, (S₁)ₘ-TPORBD₁-(S₂)ₙ-TPORBD₂-(S₃)ₚ-TPORBD₃ can comprise a sequence having at least 80%, at least 85%, at least 90%, at least 95%, at least 99%, or 100% sequence identity with SEQ ID NO: 6 or SEQ ID NO: 9.

In any of the embodiments herein, the polypeptide can comprise a sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 2 or SEQ ID NO: 10. In any of the embodiments herein, the polypeptide can comprise a signal peptide. In any of the embodiments herein, the signal peptide can comprise a sequence having at least 80%, at least 85%, at least 90%, at least 95%, at least 99%, or 100% sequence identity with SEQ ID NO: 3. In any of the embodiments herein, the polypeptide can comprise a sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 1.

In any of the embodiments herein, the polypeptide can comprise at least one, at least two, or all of the pair of intra-polypeptide disulfide bonds selected from C18-C31, C32-C45, C35-C53, C56-C71, C78-C88, C78-C96, C98-C104, C112-C121, C115-C139, C142-C157, C163-C178, C281-C341, and C387-C445, numbered according to SEQ ID NO: 2. In any of the embodiments herein, the polypeptide can form inter-polypeptide disulfide bonds at C240, C246, and/or C249, numbered according to SEQ ID NO: 2.

In some embodiments, disclosed herein is a complex comprising a dimer of the polypeptide of any of the embodiments disclosed in the present disclosure. In some embodiments, the dimer is formed via one or more inter-polypeptide disulfide bonds between two molecules of the polypeptide. In any of the embodiments herein, the polypeptide within the complex can comprise the sequence set forth in SEQ ID NO: 2 or SEQ ID NO: 10 and the complex can comprise one or more intra-polypeptide disulfide bonds selected from the group consisting of: C18-C31, C32-C45, C35-C53, C56-C71, C78-C88, C78-C96, C98-C104, C112-C121, C115-C139, C142-C157, C163-C178, C281-C341, and C387-C445, numbered according to SEQ ID NO: 2. In any of the embodiments herein, the polypeptide within the complex can comprise the sequence set forth in SEQ ID NO: 2 or SEQ ID NO: 10 and the complex can comprise one or more intra-polypeptide disulfide bonds selected from the group consisting of: C18-C31, C32-C45, C35-C53, C56-C71, C78-C88, C78-C96, C98-C115, C104-C112, C121-C139, C142-C157, C163-C178, C281-C341, and C387-C445, numbered according to SEQ ID NO: 2. In any of the embodiments herein, the complex can comprise one or more inter-polypeptide disulfide bonds selected from the group consisting of: C240-C240, C246-C246, and C249-C249, numbered according to SEQ ID NO. 2. In any of the embodiments herein, the complex can comprise a recombinant fusion protein.

In some embodiments, disclosed herein is a pharmaceutical composition comprising the polypeptide and/or the complex of any one of the embodiments disclosed in the present disclosure and a pharmaceutically acceptable carrier or excipient.

In some embodiments, disclosed herein is a kit comprising the pharmaceutical composition of any one of the embodiments disclosed in the present disclosure and instruction for using the pharmaceutical composition to treat a disease or condition.

In some embodiments, disclosed herein is a polynucleotide or an isolated nucleic acid encoding the polypeptide of any one of the embodiments disclosed in the present disclosure and/or for producing the complex of any one of the embodiments disclosed in the present disclosure. In some embodiments of the polynucleotide or isolated nucleic acid, a first nucleic acid sequence encoding the TNF binding and/or inhibiting moiety (e.g., TNFR moiety) can be in-frame with a second nucleic acid sequence encoding an immunoglobulin Fc moiety, which can be in-frame with a third nucleic acid sequence encoding the TPOR binding and/or activating moiety. In any of the embodiments herein, the isolated nucleic acid can be operably linked to a promoter sequence. In any of the embodiments herein, the isolated nucleic acid can be a DNA molecule or an RNA molecule. In any of the embodiments herein, the isolated nucleic acid can comprise an mRNA molecule such as a nucleoside-modified mRNA, a non-amplifying mRNA, a self-amplifying mRNA, or a trans-amplifying mRNA.

In some embodiments, disclosed herein is a vector comprising the isolated nucleic acid of any of the embodiments disclosed in the present disclosure. In some embodiments, disclosed herein is a particle, a virus, a virus-like structure, a cell, or a cell-like structure comprising the isolated nucleic acid and/or the vector. In any of the embodiments herein, the cell can be a mammalian cell, and the mammalian cell can be a CHO cell.

In some embodiments, disclosed herein is method of producing a recombinant fusion protein comprising a polypeptide sequence set forth in SEQ ID NO: 2 or SEQ ID NO: 10, comprising culturing the cell under conditions suitable for producing the recombinant fusion protein.

In some embodiments, disclosed herein are the polypeptide, the complex, the pharmaceutical composition, the kit, the isolated nucleic acid, the vector, and/or the particle, virus, virus-like structure, cell, or cell-like structure disclosed herein for use in the treatment of a disease or condition in a subject in need thereof, and/or for the manufacture of a medicament for treating the disease or condition.

In some embodiments, disclosed herein is a use of the polypeptide, the complex, the pharmaceutical composition, the kit, the isolated nucleic acid, the vector, and/or the particle, virus, virus-like structure, cell, or cell-like structure disclosed herein for the treating a disease or condition in a subject in need thereof.

In some embodiments, disclosed herein is a use of the polypeptide, the complex, the pharmaceutical composition, the kit, the isolated nucleic acid, the vector, and/or the particle, virus, virus-like structure, cell, or cell-like structure disclosed herein for the manufacture of a medicament for treating a disease or condition in a subject in need thereof.

In some embodiments, disclosed herein is a method for treating a disease or condition in a subject in need thereof, comprising administering an effective amount of the polypeptide, the complex, the pharmaceutical composition, the kit, the isolated nucleic acid, the vector, and/or the particle, virus, virus-like structure, cell, or cell-like structure disclosed herein to the subject.

In some embodiments, disclosed herein is a method for treating a subject in need thereof, comprising administering to the subject an effective amount of a recombinant fusion protein comprising a sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 2 or SEQ ID NO: 10.

In any of the embodiments herein, the recombinant fusion protein can comprise a dimer of a polypeptide having the sequence set forth in SEQ ID NO: 2 or SEQ ID NO: 10 or SEQ ID NO: 1.

In any of the embodiments herein, the megakaryocyte and/or platelet level in the subject can be increased following the administration of the polypeptide, the complex (e.g., the recombinant fusion protein), the pharmaceutical composition, the kit, the isolated nucleic acid, the vector, and/or the particle, virus, virus-like structure, cell, or cell-like structure disclosed herein.

In any of the embodiments herein, prior to the administration, the subject can have, be predisposed to have, or be expected to have a lower megakaryocyte and/or platelet level compared to a reference level. In any of the embodiments herein, prior to the administration, the subject can have thrombocytopenia. In some embodiments, the thrombocytopenia is caused by and/or associated with an immune disease, liver inflammations and/or damages, drug therapy, radiation therapy, and/or surgery.

In any of the embodiments herein, the thrombocytopenia can be caused by and/or associated with liver fibrosis, liver steatosis, hepatitis (for example, hepatitis B and hepatitis C), or non-alcoholic fatty liver disease (NAFLD). In any of the embodiments herein, the thrombocytopenia can be caused by and/or associated with immune thrombocytopenia (idiopathic thrombocytopenic purpura, ITP). In some embodiments, the ITP is chronic ITP. In any of the embodiments herein, the thrombocytopenia can be caused by and/or associated with a chemotherapy, immuno-oncology therapy, or combination of chemotherapy and immuno-oncology therapy, optionally wherein the immuno-oncology therapy is immune checkpoint inhibitor therapy. In any of the embodiments herein, the thrombocytopenia can be chemotherapy-induced thrombocytopenia (CIT). In any of the embodiments herein, the thrombocytopenia can be caused by and/or associated with carboplatin treatment, and/or treatment with nivolumab, pembrolizumab, dostarlimab, ipilimumab, atezolizumab, avelumab, durvalumab, or cemiplimab, or a biosimilar, bioequivalent, or biobetter thereof, or an antigen binding fragment thereof.

In any of the embodiments herein, the polypeptide, the complex (e.g., the recombinant fusion protein), the pharmaceutical composition, the isolated nucleic acid, the vector, and/or the particle, virus, virus-like structure, cell, or cell-like structure disclosed herein can be administered intravenously, intramuscularly, subcutaneously, topically, orally, transdermally, intraperitoneally, intraorbitally, by implantation, by inhalation, intrathecally, intraventricularly, or intranasally. In any of the embodiments herein, the recombinant fusion protein can be administered in a single dose or a series of doses separated by one or more intervals. In any of the embodiments herein, the recombinant fusion protein can be administered weekly. In any of the embodiments herein, the recombinant fusion protein can be administered twice a week. In any of the embodiments herein, the recombinant fusion protein is administered once every two weeks or at a longer interval. In any of the embodiments herein, the recombinant fusion protein can be administered within 24 hours of a first dose of a chemotherapy, immuno-oncology therapy, or combination of chemotherapy and immuno-oncology therapy. In any of the embodiments herein, the recombinant fusion protein can be administered at a dose from 0.01 μg/kg to 100 mg/kg based on body weight. In any of the embodiments herein, the recombinant fusion protein can be administered at a dose from 0.1 μg/kg to 10 mg/kg based on body weight. In any of the embodiments herein, the subject can have a cancer, a neoplasm, and/or an autoimmune disease. In any of the embodiments herein, platelet production in the subject can be stimulated and proliferation and/or activity of megakaryocyte-attacking cells in the subject can be downregulated. In any of the embodiments herein, proliferation and/or activity of regulatory T cells in the subject can be downregulated. In any of the embodiments herein, proliferation and/or activity of megakaryocyte-attacking cytotoxic T cells in the subject can be downregulated.

In any of the embodiments herein, the TNF binding and/or inhibiting moiety of a polypeptide disclosed herein can comprise a TNFR moiety (such as a TNFR2 moiety, e.g., an extracellular portion of human TNFR2) and/or an antibody or antigen binding fragment thereof that binds to TNF-α, wherein the thrombopoietin receptor (TPOR) binding and/or activating moiety of the polypeptide can comprise one, two, three, or more TPOR binding and/or activating domains, such as two, three, or more of a human thrombopoietin mimetic (TPM) peptide sequence (e.g., the sequence set forth in SEQ ID NO: 7).

### BRIEF DESCRIPTION OF THE DRAWINGS

FIG. 1 shows a schematic illustration of an exemplary fusion polypeptide comprising a tumor necrosis factor receptor 2 (TNFR2) moiety and a thrombopoietin receptor (TPO receptor or TPOR) binding moiety. From the N terminus to the C terminus, the fusion polypeptide rhTNFR2-Fc-TPM contains: a recombinant human TNFR2 moiety, an Fc moiety, and a TPO mimetic (TPM) moiety comprising a thrombopoietin receptor binding domain and spacers in between.

FIG. 2 shows a BaF3/c-Mpl cell growth curve in the presence of rhTNFR2-Fc-TPM (stock solution or purified product) or Nplate®. The y-axis shows cell growth analyzed by CCK-8 assay, the x-axis shows concentration of each compound.

FIG. 3 shows analysis of phosphorylation of TPOR and STAT3 in BaF3/c-Mpl cells treated with 0.5 mg/ml rhTNFR2-Fc-TPM, 0.5 mg/ml Nplate®, or a negative control.

FIG. 4 shows changes in the body weight of healthy Balb/c mice treated with a single dose of rhTNFR2-Fc-TPM at 5, 10, 50, 100, or 200 μg/kg by subcutaneous injection (“s.c”) or intravenous injection (“iv”). 50 μg/kg Nplate® was used as a positive control and PBS solution was used as a negative control (“Control”).

FIGS. 5A-5B show changes in platelet count in healthy Balb/c mice and SD rats treated with a single dose of rhTNFR2-Fc-TPM. FIG. 5A shows changes in platelet count in healthy Balb/c mice treated with a single dose of rhTNFR2-Fc-TPM at 5, 10, 50, 100, or 200 μg/kg by subcutaneous injection (“s.c”) or intravenous injection (“iv”). 50 μg/kg Nplate® was used as a positive control and PBS solution was used as a negative control (“Control”). FIG. 5B show changes in platelet count in healthy SD rats treated with a single dose of rhTNFR2-Fc-TPM compared to Nplate® or vehicle control.

FIG. 6 shows changes in the body weight of carboplatin induced CIT Balb/c mice model treated with a single dose of rhTNFR2-Fc-TPM at 100, 200, or 400 μg/kg by subcutaneous injection. 100 μg/kg Nplate® was used as a positive control and 0.9% NaCl was used as a negative control.

FIGS. 7A-7B show changes in platelet count in healthy Balb/c mice and SD rats treated with a single dose of rhTNFR2-Fc-TPM. FIG. 7A shows changes in platelet count in carboplatin induced CIT Balb/c mice model treated with a single dose of rhTNFR2-Fc-TPM at 100, 200, or 400 μg/kg by subcutaneous injection. 100 μg/kg Nplate® was used as a positive control and 0.9% NaCl was used as a negative control. FIG. 7B show changes in platelet count in the CIT mice model treated with a single dose of rhTNFR2-Fc-TPM compared to Nplate® or vehicle control.

FIGS. 8A-8B show serum concentration of rhTNFR2-Fc-TPM in SD rats and cynomolgus monkeys after a single dose of rhTNFR2-Fc-TPM subcutaneously administered at 0.02 mg/kg (low dose), 0.06 mg/kg (medium dose), or 0.2 mg/kg (high dose) or intravenously administered at 0.2 mg/kg. FIG. 8A shows serum concentration of rhTNFR2-Fc-TPM in SD rats after a single dose of rhTNFR2-Fc-TPM. FIG. 8B shows serum concentration of rhTNFR2-Fc-TPM in cynomolgus monkeys after a single dose of rhTNFR2-Fc-TPM.

FIG. 9 shows changes in platelet count in cynomolgus monkeys after a single dose of rhTNFR2-Fc-TPM subcutaneously administered at 0.02 mg/kg (low dose), 0.06 mg/kg (medium dose), or 0.2 mg/kg (high dose) or intravenously administered at 0.2 mg/kg.

FIG. 10 shows tissue distributions and excretion of ¹²⁵I labeled rhTNFR2-Fc-TPM after a single dose.

FIGS. 11A and 11B show pharmacodynamics curves after the administrations of a single dose of rhTNFR2-Fc-TPM to patient 1 and patient 2, respectively.

FIG. 12 shows pharmacokinetics curves after the administrations of a single dose of rhTNFR2-Fc-TPM to patient 1 and patient 2, respectively.

### DETAILED DESCRIPTION

Provided herein are fusion peptides or protein compositions, methods, and uses of the fusion peptides or protein for the treatment of thrombocytopenia. e.g. chemotherapy-induced thrombocytopenia (CIT), chronic immune (idiopathic) thrombocytopenic purpura (ITP), thrombocytopenia due to immune-oncology therapy, and thrombocytopenia accompanying liver inflammations or damages. In some embodiments, compositions and methods of use of fusion protein expressed by CHO cell in covalently linked dimeric forms are disclosed. In some embodiments, the resulting fusion proteins are secreted as disulfide bond-linked homo-dimers, which are more stable in structure, thereby can have longer half-life (e.g., serum half-life) than TPO in treating thrombocytopenia.

Romiplostim (Nplate®) is a TPO receptor (TPOR) agonist indicated for the treatment of chronic ITP. Thrombocytopenic purpura (ITP) is an autoimmune disease which is often treated with steroid type of hormones to suppress one's overactive immune system. Anti-TNF biologics such as etanercept (Enbrel®) have been shown to be efficacious in treating ITP (McMinn J R Jr et al., Complete recovery from refractory immune thrombocytopenic purpura in three patients treated with etanercept. Am J Hematol. 2003 June; 73(2):135-40). In some aspects, provided herein are bi-specific compositions which combine one or more TNF binding and/or antagonistic functional domains (which may suppress one's overactive immune system) with one or more TPOR binding and/or agonistic domains (which may increase platelet counts by binding to and activating the human TPO receptor).

Immune-oncology (10) drugs (e.g., anti-PD-1 antibody therapies such as Keytruda®, Opdivo®, etc.; anti-PD-L1 antibody therapies; anti-CTLA-4 antibody therapies, and/or anti-CD47 antibody therapies) could also cause platelet count decrease. For instance, more than 10% patients treated with checkpoint inhibitor therapy experienced platelet count drops and 1% with grade 3-4 severity (Shiuan E et al., Thrombocytopenia in patients with melanoma receiving immune checkpoint inhibitor therapy. J Immunother Cancer. 2017 Feb. 21; 5:8; accessdata.fda.gov/drugsatfda_docs/label/2021/125514s0961bl.pdf; and Calvo R. Hematological Side Effects of Immune Checkpoint Inhibitors: The Example of Immune-Related Thrombocytopenia. Front Pharmacol. 2019; 10:454). Additionally, CD47 has been shown to be deregulated in ITP (Catani L et al., The CD47 pathway is deregulated in human immune thrombocytopenia. Exp Hematol. 2011 April; 39(4):486-94). Since cancer patients may be treated with chemotherapy or immunotherapy or a combination of chemotherapy and immunotherapy, the bi-specific compositions disclosed herein are particularly useful in increasing platelet counts and for treating CIT and/or ITP.

Platelet count decrease could also be caused by and/or associated with liver inflammations and/or damages such as cirrhosis, liver fibrosis, liver steatosis, hepatitis (for example, hepatitis B and hepatitis C), or non-alcoholic fatty liver disease (NAFLD) (Ramadori P et al., Platelets in chronic liver disease, from bench to bedside. JHEP Rep. 2019 Oct. 25; 1(6):448-459). Provided herein are fusion peptides or protein compositions which combine TNF binding functional domains with thrombopoietin (TPO) receptor binding domains, which can be useful in increasing platelet counts and for treating symptoms of liver inflammations and/or damages.

In some aspects, provided herein is a fusion protein produced by CHO cell by recombinant DNA technology. In some embodiments, this fusion protein is a homodimer glycoprotein produced by fusing human Tumor Necrosis Factor (TNF) binding moiety to the N-terminus of human IgG1 Fc fragment, then fusing the C-terminus of the Fc fragment to a mimetic peptide comprising three thrombopoietin (TPO) receptor binding domains. In some embodiments, the TNF binding and/or inhibiting moiety on the N-terminus binds to TNF-α, thus inhibiting thrombocytopenia due to autoimmune diseases. In some embodiments, the TNF binding and/or inhibiting moiety is a chemical compound that can bind and/or inhibit TNF-α. In some embodiments, the TNF binding and/or inhibiting moiety is thalidomide or a derivative thereof, such as lenalidomide or pomalidomide. In some embodiments, the TNF binding and/or inhibiting moiety is xanthine or a derivative thereof, e.g., pentoxifylline or bupropion. In some embodiments, the TNF binding and/or inhibiting moiety is a 5-HT_{2A} agonist hallucinogen such as (R)-DOI, TCB-2, LSD, or LA-SS-Az.

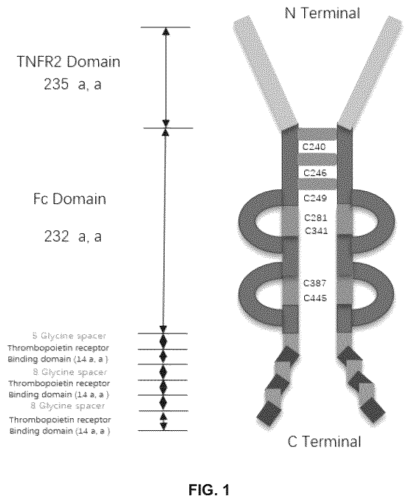

In some embodiments, the TNF binding and/or inhibiting moiety is an anti-TNF-α antibody (for example, infliximab (e.g., Remicade®) or a biosimilar, bioequivalent, or biobetter thereof, or an antigen binding fragment thereof; golimumab (e.g., Simponi®) or a biosimilar, bioequivalent, or biobetter thereof, or an antigen binding fragment thereof; adalimumab (e.g., Humira®) or a biosimilar, bioequivalent, or biobetter thereof, or an antigen binding fragment thereof; and/or certolizumab pegol (e.g., Cimzia®) or a biosimilar, bioequivalent, or biobetter thereof, or an antigen binding fragment thereof) or an antigen binding fragment thereof that binds to TNF-α. In some embodiments, the TNF binding and/or inhibiting moiety is a TNF-α receptor moiety that binds to TNF-α. In some embodiments, the TNF-α receptor moiety is a TNFR2 moiety (FIG. 1).

In some embodiments, the mimetic peptide on the C-terminus can bind to TPO receptor to induce the biological effects of endogenous TPO, e.g., to stimulate the different stages of megakaryocyte development, including expansion of precursor cells and development and maturation of polyploid megakaryocyte, thus increase platelet count. In some embodiments, a fusion protein disclosed herein can be regarded as dual functional thrombopoietin. In one aspect, a fusion protein disclosed herein can directly stimulate platelet production via binding to TPO receptors on megakaryocytes. In another aspect, a fusion protein disclosed herein can indirectly stimulate platelet production via inducing proliferation and/or activity of regulatory T cells (Tregs) and/or downregulation of proliferation and/or activity of cytotoxic T cells that attack megakaryocytes. In yet another aspect, a fusion protein disclosed herein comprise an Fc region that improves the pharmacokinetics and/or pharmacodynamics of the fusion protein, e.g., by providing slow clearance, long half-life, and/or limited tissue distribution. In some aspects, the long half-life offers the advantage of less frequent dosing in patients, e.g., as compared to small molecules. The improved pharmacokinetics and/or pharmacodynamics can also provide increased potency of a fusion protein disclosed herein as a TPO receptor agonist. As such, a fusion protein disclosed herein is not only effective in increasing platelet count via a dual mechanism, but also supports convenient dosing regimen in patients.

In some aspects, provided herein is an innovative solution to express secreted thrombopoietin mimetic protein by CHO cells. In some embodiments, the solution provided herein avoided the production of insoluble proteins by E. coli and the following complex and low efficient protein denaturation process, the complex reconstitution process, and the outdated CMC purification process, thus greatly increased the purity of the product and the stability of product quality.

In some aspects, provided herein is a thrombopoietin mimetic fusion protein having dual functional feature. In some embodiments, the thrombopoietin mimetic fusion protein provided herein has efficient thrombopoietin function on its C-terminus, in addition, its N-terminal TNF binding and/or inhibiting moiety can bind to inflammatory factor TNF-α and block TNF-α's biological function.

In some embodiments, the thrombopoietin mimetic fusion protein provided herein does not have sequence homology to endogenous TPO, thus has lower risk, e.g. compared to TPIAO® (Recombinant Human Thrombopoietin, rHuTPO), to induce autoimmune response or the production of neutralizing antibody. In some embodiments, the thrombopoietin mimetic fusion protein is suitable for long-term administration. In some embodiments, the thrombopoietin mimetic fusion protein has longer half-life compared to TPO and can be administered once a week. In some embodiments, compared to TPIAO® which requires daily administration, weekly administration of the thrombopoietin mimetic fusion protein provided herein can reduce patent sufferings during treatment and decrease treatment costs.

In some aspect, provided herein is a thrombopoietin mimetic fusion protein comprising a homodimer of two action unit linked by the Fc fragment of IgG1. In some embodiments, the C-terminus of the thrombopoietin mimetic fusion protein is linked to a TPO mimetic peptide which comprises three identical single chain units. In some embodiments, the N-terminus of the thrombopoietin mimetic fusion protein is linked to the extracellular portion of a recombinant human p75 TNF receptor. In some embodiments, the TPO mimetic peptides on the C-terminus contains 6 Thrombopoietin receptor (TPO receptor, also known as c-MPL) binding domains (SEQ ID NO.: 7) in total.

In some aspects, provided here in is a compound that binds to human tumor necrosis factor α (TNF-α) and a human thrombopoietin receptor (TPOR), wherein the compound comprises the structure: TNFR-Fc-(S₁)ₘ-TPORBD₁-(S₂)ₙ-TPORBD₂-(S₃)ₚ-TPORBD₃, wherein: TNFR is a human tumor necrosis factor receptor or a fragment or variant thereof; Fc is an Fc region of human immunoglobulin or a fragment or variant thereof; TPORBD₁, TPORBD₂, and TPORBD₃ are the same or different thrombopoietin receptor (TPOR) binding and/or activating domains; and S₁, S₂, and S₃ are spacers. In any of the embodiments herein, the lengths (e.g., amino acid sequence lengths) of the spacers. e.g., S₁, S₂, and 53, can be independently of each other. In any of the embodiments herein, the lengths of the spacers, e.g., S₁, S₂, and S₃, can be independently selected from 0 to 40 amino acid residues, for example, 0, 1, 2, 3, 4, 5, 6, 7, 8, 9, or 10 amino acid residues.

In some embodiments, the TNFR can be a recombinant human p75 TNF receptor or a fragment or variant thereof. In some embodiments, the TNFR can be the extracellular portion of a recombinant human p75 TNF receptor or a fragment or variant thereof. In some embodiments, the TNFR can comprise sequence set forth in SEQ ID NO: 4. In some embodiments, the TPORBD can bind to the human thrombopoietin receptor (TPOR).

In some embodiments, the TPORBD₁, TPORBD₂, and TPORBD₃ each can comprise a TPOR binding and/or activating domain or a fragment thereof. In some embodiments, the TPORBD₁, TPORBD₂, and TPORBD₃ can comprise different sequences. In some embodiments, the TPORBD₁, TPORBD₂, and TPORBD₃ can comprise the same sequence set forth in SEQ ID NO: 7.

In some embodiments, the Fc can be an Fc region of human IgG1 or a fragment or variant thereof. In some embodiments, the Fc can comprise sequence set forth in SEQ ID NO: 5. In some embodiments, the S₁, S₂, and S₃ can comprise different sequences. In some embodiments, the S₁, S₂, and S₃ can comprise one or more glycine residues.

In some embodiments, the compound can comprise sequence set forth in SEQ ID NO: 2 or SEQ ID NO: 10. In some embodiments, the compound can further comprise at least one pair of intra-polypeptide disulfide bonds selected from C18-C31, C32-C45, C35-C53, C56-C71, C78-C88, C78-C96, C98-C104, C112-C121, C115-C139, C142-C157, C163-C178, C281-C341, C387-C445.

In some aspects, provided herein is a complex comprising a dimer of a recombinant polypeptide, wherein the polypeptide comprises the structure: TNFR-Fc-(S₁)ₘ-TPORBD₁-(S₂)ₙ-TPORBD₂-(S₃)ₚ-TPORBD₃, wherein: TNFR is a tumor necrosis factor receptor or a fragment or variant thereof; Fc is an immunoglobulin Fc region or a fragment or variant thereof; TPORBD₁, TPORBD₂, and TPORBD₃ are the same or different thrombopoietin receptor (TPOR) binding and/or activating domains; S₁, S₂, and S₃ are the same or different spacers; and m, n, and p are 0 or greater and are integers independent of one another, wherein the recombinant polypeptides are dimerized via inter-polypeptide disulfide bonds to form the dimer.

In some embodiments, the polypeptide can comprise or consist of SEQ ID NO; 2 or SEQ ID NO: 10. In some embodiments, the recombinant polypeptides are dimerized via at least one intra-polypeptide disulfide bonds selected from the group consisting of: C18-C31, C32-C45, C35-C53, C56-C71, C78-C88, C78-C96, C98-C104, C112-C121, C115-C139, C142-C157, C163-C178, C281-C341, and C387-C445.

In some aspects, provided herein is a pharmaceutical composition comprising the thrombopoietin mimetic fusion protein and a pharmaceutically acceptable carrier.

In some embodiments, provided herein is a method for increasing megakaryocytes or platelets in a patient in need thereof, comprising administering to the patient an effective amount of the thrombopoietin mimetic fusion protein comprising SEQ ID NO: 2 or SEQ ID NO: 10. In some embodiments, provided herein is a method of treating thrombocytopenia in a patient in need thereof, comprising administering to the patient an effective amount of the thrombopoietin mimetic fusion protein comprising SEQ ID NO: 2 or SEQ ID NO: 10.

In some embodiments, the thrombocytopenia can be caused by autoimmune diseases, liver inflammations and/or damages, or induced by drug therapy, radiation therapy, or surgery. In some embodiments, the thrombocytopenia induced by autoimmune diseases can be chronic immune (idiopathic) thrombocytopenic purpura (ITP). In some embodiments, the CD47 pathway is deregulated in ITP. In some embodiments, the liver inflammations and/or damages can be cirrhosis, liver fibrosis, liver steatosis, hepatitis (for example, hepatitis B and hepatitis C), or non-alcoholic fatty liver disease (NAFLD). In some embodiments, the drug therapy can be chemotherapy. In some embodiments, the chemotherapy can be carboplatin, wherein the thrombocytopenia can be carboplatin-induced. In some embodiments, the thrombocytopenia induced by drug therapy can be chemotherapy-induced thrombocytopenia (CIT). In some embodiments, the drug therapy can be immune-oncology therapy. In some embodiments, the immune-oncology therapy can be immune checkpoint inhibitor therapy. In some embodiments, the immune checkpoint inhibitor therapy can inhibit CD47, CTLA-4, PD-1 and/or PD-L1 (for example, nivolumab, pembrolizumab, dostarlimab, ipilimumab, atezolizumab, avelumab, durvalumab, or cemiplimab, or a biosimilar, bioequivalent, or biobetter thereof, or an antigen binding fragment thereof). In some embodiments, the immune checkpoint inhibitor therapy can be combined with chemical and/or radiation therapies.

In some embodiments, the rhTNFR2-Fc-TPM fusion protein can be administered via subcutaneous injection. In some embodiments, the rhTNFR2-Fc-TPM fusion protein can be administered via intravenous injection. In some embodiments, the rhTNFR2-Fc-TPM fusion protein can be administered in a single dose or a series of doses separated by intervals of once per week or twice per week. In some embodiments, the rhTNFR2-Fc-TPM fusion protein can be administered from 0.1 μg/kg to 100 mg/kg. In some embodiments, the rhTNFR2-Fc-TPM fusion protein can be administered from 1 μg/kg to 100 mg/kg. In some embodiments, wherein the rhTNFR2-Fc-TPM fusion protein can be administered weekly. In some embodiments, the rhTNFR2-Fc-TPM fusion protein can be administered twice a week. In some embodiments, the first dose of the rhTNFR2-Fc-TPM fusion protein can be administered within 24 hours of the first dose of chemotherapy.

In some aspects, provided herein is a polynucleotide encoding the amino acid sequence set forth in any of the sequences selected from SEQ ID NOs: 1-7 and 9. In some aspects, provided herein is a vector comprising the polynucleotide encoding the amino acid sequence set forth in any of the sequences selected from SEQ ID NOs: 1-7 and 9.

In some aspects, provided herein is a host cell comprising the vector comprising the polynucleotide encoding the amino acid sequence set forth in any of the sequences selected from SEQ ID NOs: 1-7 and 9. In some embodiments, the host cell can be selected from a bacterial, yeast, fungi, insect, plant or mammalian cell. In some embodiments, the host cell can be a mammalian cell. In some embodiments, the host cell can be a CHO cell.

In some embodiments, provided herein is a method of making a rhTNFR2-Fc-TPM fusion protein, comprising culturing the host cell comprising the polynucleotide encoding the amino acid sequence set forth in any of the sequences selected from SEQ ID NOs: 1-7 and 9, under conditions suitable for producing the rhTNFR2-Fc-TPM fusion protein.

Also provided herein are composition with thrombopoietin activity comprising the proteins provided herein, methods of producing the fusion proteins provided herein, methods of treating subjects with the fusion proteins and compositions provided herein, and kits.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### 1. TNF-α Binding Domain

The proteins provided herein comprise an N-terminal domain that can bind to inflammatory factor Tumor Necrosis Factor alpha (TNF-α) and block TNF-α's biological function. In some embodiments, the TNF-α binding moiety is a chemical compound. In some embodiments, the TNF-α binding moiety is an anti-TNF-α antibody (for example, infliximab (e.g., Remicade®) or a biosimilar, bioequivalent, or biobetter thereof, or an antigen binding fragment thereof; golimumab (e.g., Simponi®) or a biosimilar, bioequivalent, or biobetter thereof, or an antigen binding fragment thereof; adalimumab (e.g., Humira®) or a biosimilar, bioequivalent, or biobetter thereof, or an antigen binding fragment thereof; and/or certolizumab pegol (e.g., Cimzia®) or a biosimilar, bioequivalent, or biobetter thereof, or an antigen binding fragment thereof) or an antigen binding fragment thereof that binds to TNF-α. In some embodiments, the TNF binding and/or inhibiting moiety is a TNF-α receptor moiety that binds to TNF-α. In some embodiments, the TNF-α receptor moiety is TNFR2 moiety. In some embodiments, blocking TNF-α's biological function can treat or reduce the chronic immune (idiopathic) thrombocytopenic purpura (ITP).

TNF-α is an inflammatory cytokine produced by macrophages/monocytes during acute inflammation and is responsible for a diverse range of signaling events within cells, leading to necrosis or apoptosis. The protein is also important for resistance to infection and cancers. TNF alpha exerts many of its effects by binding, as a trimer, to either TNFR1, which is a 55 kDa cell membrane receptor, or to TNFR2, which is a 75 kDa cell membrane receptor.

ITP is hematologic disorder caused by autoimmune opsonization and premature destruction of platelets. Pro-inflammatory cytokines IL-2. TNF-α, and IFN-γ are secreted following a Th1 response and are elevated in patients with chronic ITP. TNF-α can upregulate the activity of phagocytes and can cause ITP (Wajant H and Siegmund D (2019) TNFR1 and TNFR2 in the Control of the Life and Death Balance of Macrophages. Front. Cell Dev. Biol. 7:91). Thus, the identification and normalization of apoptotic pathways and TNF-α receptors are potential therapeutic approaches for ITP.

Recent findings of the clinical studies indicated that inhibition of TNF-α may be able to promote platelet elevation and is well tolerated. Thus, the dual targets design of rhTNFR2-Fc-TPM is very promising in the development of treatments for chemotherapy-induced thrombocytopenia (CIT).

In some cases, the N-terminal domain of the fusion peptide or protein is a Tumor Necrosis Factor Receptor (TNFR) or a portion thereof. In some embodiments, the N-terminal domain of the fusion peptide or protein is TNFR2 or a portion thereof. In some embodiments, the N-terminal domain of the fusion peptide or protein comprises N-terminal truncation compared to human native TNFR2.

In some cases, the N-terminal domain of the fusion peptide or protein is a TNFR2 domain comprising human TNFR2 or a fragment or variant thereof. In some embodiments, the TNFR2 domain does not contain the signal peptide of native human TNFR2. In some embodiments, the TNFR2 domain does not contain the transmembrane domain of native human TNFR2. In some embodiments, the TNFR2 domain does not contain the cytoplasmic domain of native human TNFR2. In some embodiments, the TNFR2 domain is the extracellular portion of a recombinant human TNFR2 receptor.

In some cases, the TNFR2 domain of the fusion protein comprises one or more amino acid substitutions, deletions, or insertions compared to a native human TNFR sequence. In some embodiments, the TNFR2 domain of the fusion protein comprises substitution at one or more amino acid positons compared to a native human TNFR sequence. In some embodiments, amino acid substitutions, deletions, or insertions could result in improvement of expression, purification, or stability profile of the peptide or protein. In some embodiments, amino acid substitutions, deletions, or insertions could result in improvement of binding affinity and specificity profile of the peptide or protein.

In some embodiments, the TNFR2 domain of the fusion peptide or protein comprises the sequence set forth in SEQ ID NO: 4. In some embodiments, the TNFR peptide comprises an amino acid sequence having at least or about 80%, 85%, 90%, 92%, 95%, or 97% sequence identity to SEQ ID NO: 4 shown below.

SEQ ID NO. 4:
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDVVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGD

In some embodiments, the TNFR2 domain of the fusion peptide or protein comprises a sequence that is different from CRPGFGVARP. In some embodiments, the TNFR2 domain of the fusion peptide or protein comprises VLNCTARTEL instead of CRPGFGVARP.

In some embodiments, the TNFR2 domain of the fusion peptide or protein comprises one or more intra-polypeptide disulfide bonds selected from: C18-C31, C32-C45, C35-C53, C56-C71, C78-C88, C78-C96, C98-C104, C112-C121, C115-C139, C142-C157, C163-C178 (numbering based on SEQ ID NO; 4).

### II. TPO Mimetic Domain

Thrombopoietin receptor (TPOR, also known as C-MPL) is expressed on the surface of stem cells, megakaryocytes, and megakaryocyte precursors. Stimulation of TPOR can activate Janus Activating Kinase 2/Signal Transducers and Activators of Transcription 5 (JAK2/STAT5) signaling pathway and change gene expression levels, which in turn promotes the differentiation of stem cells into megakaryocyte pathway, promotes expansion and differentiation of human bone marrow progenitor cells, increase the level of mature megakaryocytes, and finally promotes the formation of platelets and their release into peripheral circulation.

In some cases, the C-terminal domain of the fusion peptide or protein comprises a thrombopoietin mimetic (TPM) domain. In some embodiments, the C-terminal domain of the fusion peptide or protein comprises two or more TPM domains. In some embodiments, the C-terminal domain of the fusion peptide or protein comprises three TPM domains. In some embodiments, the two or more TPM domains have different amino acid sequences. In some embodiments, the two or more TPM domains have the same amino acid sequence. In some embodiments, the TPM domain does not have sequence homology to human native TPO.

In some embodiments, the TPM domain comprises the sequence set forth in SEQ ID NO: 7. In some embodiments, the TPM domain comprises an amino acid sequence having at least or about 80%, 85%, 90%, 92%, 95%, or 97% sequence identity to SEQ ID NO: 7 shown below.

SEQ ID NO. 7: IEGPTLRQWLAARA

In some cases, the TPM domain of the fusion peptide or protein comprises multiple TPM domains and spacers in between. In some embodiments, the TPM domain of the fusion peptide or protein comprises sequence set forth in SEQ ID NO: 6. In some embodiments, the TPM domain of the fusion peptide or protein comprises an amino acid sequence having at least or about 80%, 85%, 90%, 92%, 95%, or 97% sequence identity to SEQ ID NO: 6 shown below.

SEQ ID NO. 6:
GGGGGIEGPTLRQWLAARAGGGGGGGGIEGPTLRQWLAARAGGGGGGGG
IEGPTLRQWLAARA

In some cases, two or more TPM domains are separated by spacer sequences. In some embodiments, the spacer sequences comprises 1, 2, 3, 4, 5, 6, 7, 8, 9, or 10 amino acids. In some embodiments, the spacer sequences comprises at least one glycine residue. In some embodiments, the spacer sequences comprises 1, 2, 3, 4, 5, 6, 7, 8, 9, or 10 glycine residues. In some embodiments, the spacer length between each TPM domain are different. In some embodiments, the spacer length between each TPM domain are the same. In some embodiments, the spacer amino acid sequences between each TPM domain are different. In some embodiments, the spacer amino acid sequences between each TPM domain are the same.

The term “spacer” refers to a moiety (e.g., a polyethylene glycol (PEG) polymer) or an amino acid sequence (e.g., a 1-200 amino acid sequence) occurring between two elements, e.g., peptides or protein domains, to provide space and/or flexibility between the two elements. An amino acid spacer is part of the primary sequence of a polypeptide (e.g., fused to the spaced peptides via the polypeptide backbone). The formation of disulfide bonds, e.g., between two hinge regions that form an Fc domain, is not considered a linker.

In some embodiments, the amino acid spacers are independently selected from: GG,

GGG,
GGGG,
GGGGG,
GGGGGG,
GGGGGGGG,
GGGA,
GGGG,
GGGAG,
GGGAGG,
GGGAGGG,
GGGS,
GGGGA,
GGGGS,
GGAG,
GGSG,
AGGG,
SGGG,
GAGA,
GSGS,
GAGAGA,
GSGSGS,
GAGAGAGA,
GSGSGSGS,
GAGAGAGAGA,
GSGSGSGSGS,
GAGAGAGAGAGA,
GSGSGSGSGSGS,
GGAGGA,
GGSGGS,
GGAGGAGGA,
GGSGGSGGS,
GGAGGAGGAGGA,
GGSGGSGGSGGS,
GGAGGGAG,
GGSGGGSG,
GGAGGGAGGGAG,
and
GGSGGGSGGGSG,
GGGGAGGGGAGGGGA,
GGGGSGGGGSGGGGS,
AAAL,
AAAK,
AAAR,
EGKSSGSGSESKST,
GSAGSAAGSGEF,
AEAAAKEAAAKA,
KESGSVSSEQLAQFRSLD,
GENLYFQSGG,
SACYCELS,
RSIAT,
RPACKIPNDLKQKVMNH,
GGSAGGSGSGSSGGSSGASGTGTAGGTGSGSGTGSG,
AAANSSIDLISVPVDSR,
GGSGGGSEGGGSEGGGSEGGGSEGGGSEGGGSGGGS,
EAAAK,
and
PAPAP.

### III. Fc Domain

In some embodiments, a polypeptide described herein may include an Fc domain monomer of an immunoglobulin or a fragment of an Fc domain to increase the serum half-life of the polypeptide. A polypeptide described herein may form a dimer (e.g., homodimer or heterodimer) through the interaction between two Fc domain monomers, which form an Fc domain in the dimer. As conventionally known in the art, an Fc domain is the protein structure that is found at the C-terminus of an immunoglobulin. An Fc domain includes two Fc domain monomers that are dimerized by the interaction between the C_{H}3 antibody constant domains. A wild-type Fc domain forms the minimum structure that binds to an Fc receptor, e.g., FcγRI, FcγRIIa, FcγRIIb, FcγRIIIa, FcγRIIIb, FcγRIV. In some embodiments, an Fc domain may be mutated to lack effector functions, typical of a “dead” Fc domain. For example, an Fc domain may include specific amino acid substitutions that are known to minimize the interaction between the Fc domain and an Fcγ receptor. In some embodiments, an Fc domain is from an IgG1 antibody and includes amino acid substitutions L234A, L235A, and G237A. In some embodiments, an Fc domain is from an IgG1 antibody and includes amino acid substitutions D265A, K322A, and N434A. The aforementioned amino acid positions are defined according to Kabat (Sequences of Proteins of Immunological Interest, 5th Ed. Public Health Service, National Institutes of Health. Bethesda, Md. (1991)). The Kabat numbering of amino acid residues may be determined for a given antibody by alignment at regions of homology of the sequence of the antibody with a “standard” Kabat numbered sequence. Furthermore, in some embodiments, an Fc domain does not induce any immune system-related response. For example, the Fc domain in a dimer of a polypeptide may be modified to reduce the interaction or binding between the Fc domain and an Fcγ receptor.

In some cases, TNF-α binding domain and the TPO mimetic domain of fusion peptide or protein is joined together by an Fc domain comprising the Fc region of a human immunoglobulin or a fragment or variant thereof. In some embodiments, the Fc domain of the fusion peptide or protein is an Fc region of human IgG1 or a fragment or variant thereof. In some embodiments, the Fc domain of the fusion peptide or protein has a N-terminal truncation compared to native human immunoglobulin Fc region. In some embodiments, the Fc domain of the fusion peptide or protein does not contain the C_{H}1 domain of native human immunoglobulin Fc region.

In some cases, the Fc domain of the fusion peptide or protein comprises one or more amino acid substitutions, deletions, or insertions compared to a native human IgG1 sequence. In some embodiments, the Fc domain of the fusion protein comprises substitution at one or more amino acid positons in the C_{H}2 domain. In some embodiments, the Fc domain of the fusion protein comprises substitution at one or more amino acid positons in the DE-turn. In some embodiments, the Fc domain of the fusion protein comprises a substitution of the naturally occurring amino acid at position 297 wherein said substitution detectably reduces and/or abrogates glycosylation at position 297. In specific embodiments, the Fc domain of the fusion protein comprises a substitution of cysteine for asparagine at position 297 of the heavy chain of the antibody. In yet other embodiments, the Fc domain of the fusion protein lacks glycosylation at position 297. In some embodiments, the Fc domain of the fusion protein comprises a N297Q substitution which corresponds to N317 in SEQ ID NO: 2. In each of these, the numbering system of the constant region is that of the EU index as set forth in Kabat.

In some embodiments, amino acid substitutions, deletions, or insertions could result in improvement of expression, purification, or stability profile of the fusion peptide or protein. In some embodiments, amino acid substitutions, deletions, or insertions could result in improvement of binding affinity and specificity profile of the fusion peptide or protein.

In some embodiments, the Fc domain of the fusion peptide or protein comprises the sequence set forth in SEQ ID NO: 5. In some embodiments, the Fc domain of the fusion peptide or protein comprises an amino acid sequence having at least or about 80%, 85%, 90%, 92%, 95%, or 97% sequence identity to SEQ ID NO: 5 shown below.

SEQ ID NO. 5:
EPKSCDKTHTCPPCPAPELLGGPSVFLFPPKPKDTLMISRTPEVTCVV
VDVSHEDPEVKFNWYVDGVEVHNAKTKPREEQYQSTYRVVSVLTVLHQ
DWLNGKEYKCKVSNKALPAPIEKTISKAKGQPREPQVYTLPPSRDELT
KNQVSLTCLVKGFYPSDIAVEWESNGQPENNYKTTPPVLDSDGSFFLY
SKLTVDKSRWQQGNVFSCSVMHEALHNHYTQKSLSLSPGK

In some cases, the Fc domain of the fusion peptide or protein comprises one or more disulfide bonds. In some embodiments, the Fc domain of the fusion peptide or protein comprises one or more intra-chain disulfide bonds selected from: C281-C341 and C387-C445 (numbering based on SEQ ID NO: 2). In some embodiments, two of the fusion peptide forms a dimer comprising one or more inter-chain disulfide bonds selected from: C240-C240, C246-C246, and C249-249 (numbering based on SEQ ID NO: 2).

### IV. Fusion Peptides and Proteins

As used herein, the term “fused” is used to describe the combination or attachment of two or more elements, components, or protein domains, e.g., peptides or polypeptides, by means including chemical conjugation, recombinant means, and chemical bonds, e.g., amide bonds. For example, two single peptides in tandem series can be fused to form one contiguous protein structure, e.g., a polypeptide, through chemical conjugation, a chemical bond, a peptide linker, or any other means of covalent linkage. In some embodiments of a polypeptide described herein, a TNF binding and/or inhibiting moiety (for example, a chemical compound that could bind to TNF-α, an anti-TNF-α antibody or an antigen binding fragment thereof, or an extracellular TNFR2 sequence) may be fused to the N-terminus of a moiety (e.g., Fc domain monomer, a wild-type Fc domain, or an Fc domain with amino acid substitutions) by way of a linker or through a chemical bond, e.g., a peptide bond. In some embodiments of a polypeptide described herein, the moiety (e.g., Fc domain monomer, a wild-type Fc domain, or an Fc domain with amino acid substitutions) may be fused to the N-terminus of a TPO mimetic peptide by way of a linker or through a chemical bond, e.g., a peptide bond.

In some embodiments, provided herein is a fusion peptide comprising from amino to carboxyl terminus: a) a first region comprising a human TNFR2 receptor or fragment or variant thereof; b) a second region comprising human IgG Fc region or fragment or variant thereof, and c) a TPO mimetic peptide. In some embodiments, the TPO mimetic peptide comprises more than one TPOR binding and/or activating domain. In some embodiments, the multiple TPOR binding and/or activating domains are linked to each other and the Fc region by one or more peptide linkers. In some embodiments, the peptide linkers are glycine linkers, for example, a 5 amino acid glycine peptide linker.

In some embodiments, the recombinant polypeptide is or comprises the sequence set forth in SEQ ID NO: 2 or SEQ ID NO: 10. In some embodiments, the recombinant poly peptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 2 or SEQ ID NO: 10.

In some embodiments, the above fusion polypeptide may comprise an N-terminal signal peptide provided in SEQ ID NO: 3. In some embodiments, the recombinant polypeptide is or comprises the sequence set forth in SEQ ID NO: 1. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1.

In some embodiments, the N-terminal TNFR2 domain of the fusion polypeptides form inter-polypeptide disulfide bonds. In some embodiments, the Fc domain of the fusion polypeptides form inter-polypeptide disulfide bonds. In some embodiments, the intra-polypeptide disulfide bonds may comprise one or more or all of C18-C31, C32-C45, C35-C53, C56-C71, C78-C88, C78-C96, C98-C104, C112-C121, C115-C139, C142-C157, C163-C178, C281-C341, and C387-C445, in any suitable combination.

In some embodiments, provided herein is a fusion dimer protein comprising two fusion polypeptides, each fusion polypeptide comprising, from amino to carboxyl terminus: a) a first region comprising a human TNFR2 receptor or fragment or variant thereof; b) a second region comprising human IgG Fc region or fragment or variant thereof; and c) a TPO mimetic peptide, wherein the Fc of the fusion polypeptides form inter-polypeptide disulfide bonds. In some embodiments, the inter-polypeptide disulfide bonds may comprise one or more or all of C240-C240, C246-C246, and C249-249, in any suitable combination.

In some embodiments, the fusion polypeptide in the dimer may comprise one or more glycosylation sites (e.g., N-glycosylation), for example, at one or both of N149 and N171 (numbering based on SEQ ID NO: 2). N-linked glycosylation is the attachment of an oligosaccharide (sometimes also referred to as glycan) to a nitrogen atom (e.g., the amide nitrogen of an asparagine (Asn, N) residue of a protein). In some embodiments, to avoid any potential of ADCC mediated by N-linked glycosylation at N317 (numbering based on SEQ ID NO: 2) from human IgG Fc, N317 can be mutated, e.g., to glutamine (Gln, Q). In some embodiments, a fusion polypeptide herein can be produced in a host cell (e.g., E. coli) where there is no glycan. In some embodiments, the fusion polypeptide in the dimer may comprise one or more deamination modification sites, for example, at one or more or of Q82, Q109, N306, Q317, and Q524 (numbering based on SEQ ID NO: 2), in any suitable combination. In some embodiments, the fusion polypeptide in the dimer may comprise one or more oxidative modification sites, for example, at one or both of M30 and M272 (numbering based on SEQ ID NO: 2). In some embodiments, the fusion polypeptide in the dimer may comprise aspartic acid isomerization modification sites, for example, at D300 (numbering based on SEQ ID NO: 2). In some embodiments, the fusion polypeptide herein can comprise any of the amino acid residues disclosed herein in any suitable combination.

### V. Polynucleotides, Vectors, and Method of Producing Recombination Peptides and Proteins

Also provided are polynucleotides (nucleic acid molecules) encoding the fusion polypeptides provided herein, vectors for genetically engineering cells to express such fusion polypeptides, and host cell comprising the polynucleotides or vectors for genetically engineering cells to express such fusion polypeptides.

In some embodiments, provided are polynucleotides that encode fusion polypeptides provided herein. In some aspects, the polynucleotide contains a single nucleic acid sequence, such as a nucleic acid sequence encoding a fusion polypeptide. In some embodiments, the polynucleotide encoding the fusion polypeptide contains at least one promoter that is operatively linked to control expression of the fusion polypeptide.

In one aspect, current disclosure provides an isolated nucleic acid molecule that comprises a nucleotide sequence encoding an amino acid sequence of a fusion peptide provided by the present disclosure. The amino acid sequence encoded by the nucleotide sequence may be any portion of the fusion protein describe herein, such as the TNFR2 domain, the Fc domain, the TPM domain, the full length fusion peptide, or may be the full length fusion peptide with N-terminal signal peptide. A nucleic acid of the disclosure can be, for example, DNA or RNA, and may or may not contain intronic sequences. Typically, the nucleic acid is a cDNA molecule.

In other embodiments, the nucleic acid molecule comprises or consists of a nucleotide sequence that encodes an amino acid sequence as set forth in any one of SEQ ID NOs: 1-7 and 9.

The present disclosure further provides a vector that comprises a nucleic acid molecule provided by the present disclosure. The nucleic acid molecule may encode any portion of the fusion protein describe herein, such as the TNFR2 domain, the Fc domain, the TPM domain, the full length fusion peptide, or may be the full length fusion peptide with N-terminal signal peptide.

To express a fusion protein of the disclosure. DNAs encoding the fusion protein are inserted into expression vectors such that the DNA molecules are operatively linked to transcriptional and translational control sequences. In this context, the term “operatively linked” means that a DNA molecule encoding the fusion peptide is ligated into a vector such that transcriptional and translational control sequences within the vector serve their intended function of regulating the transcription and translation of the DNA molecule. The expression vector and expression control sequences are chosen to be compatible with the expression host cell used. The DNA molecule encoding the fusion peptide is inserted into the expression vector by any suitable methods (e.g., ligation of complementary restriction sites on the DNA molecule encoding the fusion peptide and vector, or homologous recombination-based DNA ligation). Additionally or alternatively, the recombinant expression vector can encode a signal peptide that facilitates secretion of the fusion peptide from a host cell. The antibody chain gene can be cloned into the vector such that the signal peptide is linked in-frame to the amino terminus of the DNA molecule encoding the fusion peptide. In some embodiments, the nucleic acid sequence encoding the signal peptide comprise a nucleotide sequence that encodes an amino acid sequence as set forth in SEQ ID NO: 3.

In addition to the nucleic acid sequence encoding the fusion peptide, the expression vectors of the disclosure typically carry regulatory sequences that control the expression of the nucleic acid sequence encoding the fusion peptide in a host cell. The term “regulatory sequence” is intended to include promoters, enhancers and other expression control elements (e.g., polyadenylation signals) that control the transcription or translation of the nucleic acid sequence encoding the fusion peptide. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Examples of regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or R-globin promoter. Still further, regulatory elements composed of sequences from different sources, such as the SR promoter system, which contains sequences from the SV40 early promoter and the long terminal repeat of human T cell leukemia virus type 1 (Takebe, Y. et al. (1988) Mol. Cell. Biol. 8:466-472).

In addition to the nucleic acid sequence encoding the fusion peptide and regulatory sequences, the expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the fusion peptide, the expression vector encoding the fusion peptide is transfected into a host cell by any suitable techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the fusion peptide of the disclosure in either prokaryotic or eukaryotic host cells, expression of antibodies in eukaryotic cells, and typically mammalian host cells, is most typical.

The present disclosure further provides a host cell containing a nucleic acid molecule provided by the present disclosure. The host cell can be virtually any cell for which expression vectors are available. It may be, for example, a higher eukaryotic host cell, such as a mammalian cell, a lower eukaryotic host cell, such as a yeast cell, and may be a prokaryotic cell, such as a bacterial cell. Introduction of the recombinant nucleic acid construct into the host cell can be effected by calcium phosphate transfection, DEAE, dextran mediated transfection, electroporation or phage infection.

Suitable prokaryotic hosts for transformation include E. coli, Bacillus subtilis, Salmonella typhimurium and various species within the genera Pseudomonas, Streptomyces, and Staphylococcus.

Mammalian host cells for expressing a fusion peptide of the disclosure include, for example, Chinese Hamster Ovary (CHO) cells (including dhfr-CHO cells, described in Urlaub and Chasin, Proc. Natl. Acad Sci. USA 77:4216-4220 (1980), used with a DHFR selectable marker, e.g., as described in Kaufman and Sharp, J. Mol. Biol. 159:601-621 (1982), NS0 myeloma cells, COS cells and Sp2 cells. In particular, for use with NS0 myeloma or CHO cells, another expression system is the GS (glutamine synthetase) gene expression system disclosed in WO 87/04462, WO 89/01036 and EP 338,841. When expression vectors comprising the nucleic acid sequence encoding the fusion peptide are introduced into mammalian host cells, the fusion peptide is produced by culturing the host cells for a period of time sufficient to allow for expression of the fusion peptide in the host cells or secretion of the antibody into the culture medium in which the host cells are grown. The fusion peptide can be recovered from the culture medium using any suitable protein purification methods.

VI. Compositions and Formulations with Thrombopoietin Activity

In some embodiments, provided herein is a pharmaceutical composition comprising the dimer of the rhTNFR2-Fc-TPM fusion peptides having the sequence set forth in SEQ ID NO: 2. In some embodiments, provided herein is a pharmaceutical composition comprising the dimer of the rhTNFR2-Fc-TPM fusion peptides having the sequence set forth in SEQ ID NO: 10. In some embodiments, the pharmaceutical composition comprises dimerized fusion polypeptides provided herein, and optionally a pharmaceutically acceptable carrier.

The terms “pharmaceutically acceptable carrier” or “pharmaceutically acceptable excipient” refer to any inactive substance that is suitable for use in a formulation for the delivery of a binding molecule. A carrier may be an antiadherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), polysorbate 20, dextrose, vegetable oils (such as olive oil), saline, buffer, buffered saline, and isotonic agents such as sugars, polyalcohols, sorbitol, and sodium chloride.

The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering a binding molecule is a sterile liquid, such as a solution, suspension, or dispersion, for injection or infusion. Sterile solutions can be prepared by incorporating the rhTNFR2-Fc-TPM fusion protein in the required amount in an appropriate carrier, followed by sterilization microfiltration. Generally, dispersions are prepared by incorporating the rhTNFR2-Fc-TPM fusion protein into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

The relative amount of rhTNFR2-Fc-TPM fusion protein included in the composition will vary depending upon a number of factors, such as the carriers used, dosage form and desired release and pharmacodynamic characteristics. The amount of rhTNFR2-Fc-TPM fusion protein in a single dosage form will generally be that amount which produces a therapeutic effect, but may also be a lesser amount. Generally, this amount will range from about 0.01 percent to about 99 percent, from about 0.1 percent to about 70 percent, or from about 1 percent to about 30 percent relative to the total weight of the dosage form.

In some embodiments, the composition with thrombopoietin activity comprise pharmaceutically acceptable carriers including for instance, solvents, bulking agents, buffering agents, tonicity adjusting agents, and preservatives (Pramanick et al., Pharma Times, 45:65-77, 2013). In some embodiments the composition with thrombopoietin activity may comprise an carrier that functions as one or more of a solvent, a bulking agent, a buffering agent, and a tonicity adjusting agent (e.g., sodium chloride in saline may serve as both an aqueous vehicle and a tonicity adjusting agent).

In some embodiments, the composition with thrombopoietin activity comprise an aqueous vehicle as a solvent. Suitable vehicles include for instance sterile water, saline solution, phosphate buffered saline, and Ringer's solution. In some embodiments, the composition is isotonic.

The composition with thrombopoietin activity may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine or pharmaceutically acceptable salts thereof. The buffering agent may further comprise hydrochloric acid or sodium hydroxide. In some embodiments, the buffering agent maintains the pH of the composition within a range of 5 to 8. In some embodiments, the pH is greater than (lower limit) 5 or 6. In some embodiments, the pH is less than (upper limit) 8, or 7. That is, the pH is in the range of from about 5 to 8 in which the lower limit is less than the upper limit.

The composition with thrombopoietin activity may comprise a tonicity adjusting agent. Suitable tonicity adjusting agents include for instance dextrose, sucrose, glycerol, sodium chloride, glycerin and mannitol.

The composition with thrombopoietin activity may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The composition with thrombopoietin activity may comprise a preservative. Suitable preservatives include for instance antioxidants and antimicrobial agents. However, in preferred embodiments, the composition with thrombopoietin activity is prepared under sterile conditions and is in a single use container, and thus does not necessitate inclusion of a preservative.

In some embodiments, the composition can be provided as a sterile composition. The pharmaceutical composition typically contains an effective amount of a disclosed fusion protein and can be prepared by conventional techniques. Typically, the amount of fusion protein in each dose of the immunogenic composition is selected as an amount which induces increase of platelet counts without significant, adverse side effects. In some embodiments, the composition can be provided in unit dosage form for use to induce increase of platelet counts in a subject. A unit dosage form contains a suitable single preselected dosage for administration to a subject, or suitable marked or measured multiples of two or more preselected unit dosages, and/or a metering mechanism for administering the unit dose or multiples thereof.

### VII. Methods of Treating Thrombocytopenia

In some embodiments, provided herein is a method for treating thrombocytopenia, comprising administering to the patient an effective amount of an rhTNFR2-Fc-TPM fusion protein comprising SEQ ID NO: 2. In some embodiments, provided herein is a method for treating thrombocytopenia, comprising administering to the patient an effective amount of an rhTNFR2-Fc-TPM fusion protein comprising SEQ ID NO: 10.

In some embodiments, the thrombocytopenia can be caused by autoimmune diseases, liver inflammations and/or damages, or induced by drug therapy, radiation therapy, or surgery. In some embodiments, the thrombocytopenia induced by autoimmune diseases can be chronic immune (idiopathic) thrombocytopenic purpura (ITP). In some embodiments, the CD47 pathway is deregulated in ITP. In some embodiments, the liver inflammations and/or damages can be cirrhosis, liver fibrosis, liver steatosis, hepatitis (for example, hepatitis B and hepatitis C), or non-alcoholic fatty liver disease (NAFLD). In some embodiments, the drug therapy can be chemotherapy. In some embodiments, the chemotherapy can be carboplatin, wherein the thrombocytopenia can be carboplatin-induced. In some embodiments, the thrombocytopenia induced by drug therapy can be chemotherapy-induced thrombocytopenia (CIT). In some embodiments, the drug therapy can be immune-oncology therapy. In some embodiments, the immune-oncology therapy can be immune checkpoint inhibitor therapy. In some embodiments, the immune checkpoint inhibitor therapy can inhibit CD47, CTLA-4, PD-1 and/or PD-L1 (for example, nivolumab, pembrolizumab, dostarlimab, ipilimumab, atezolizumab, avelumab, durvalumab, or cemiplimab, or a biosimilar, bioequivalent, or biobetter thereof, or an antigen binding fragment thereof). In some embodiments, the immune checkpoint inhibitor therapy can be combined with chemical and/or radiation therapies.

In some embodiments, the rhTNFR2-Fc-TPM fusion protein is administered via subcutaneous injection. In some embodiments, the rhTNFR2-Fc-TPM fusion protein is administered via intravenous injection. In some embodiments, rhTNFR2-Fc-TPM fusion protein is administered in a single dose or a series of doses separated by intervals of once per week or twice per week, or once every two weeks or once every one and half weeks, or at a longer interval. In some embodiments, the rhTNFR2-Fc-TPM fusion protein is administered weekly. In some embodiments, the rhTNFR2-Fc-TPM fusion protein is administered twice a week. In some embodiments, the rhTNFR2-Fc-TPM fusion protein is administered once every two weeks or once every one and half weeks, or at a longer interval. In some embodiments, the rhTNFR2-Fc-TPM fusion protein is administered once every one and half weeks, once every two weeks, once every two and half weeks, once every 3, 3.5, 4, 4.5, 5, 5.5, 6, 6.5, 7, 7.5, 8, 8.5, 9, 9.5, 10 or 10.5 weeks. In some embodiments, the rhTNFR2-Fc-TPM fusion protein is administered once every two weeks. In some embodiments, the rhTNFR2-Fc-TPM fusion protein is administered once every three weeks. In some embodiments, the rhTNFR2-Fc-TPM fusion protein is administered once every four weeks.

In some aspect, administration of the fusion peptide or protein is coordinated with the chemotherapy or immuno-oncology therapy cycles the subject maybe receiving. In some embodiments, the first dose of the rhTNFR2-Fc-TPM fusion protein is administered within 24 hours of the first dose of chemotherapy immuno-oncology therapy. In some embodiments, the first dose of the rhTNFR2-Fc-TPM fusion protein is administered within 48 hours of the first dose of chemotherapy immuno-oncology therapy. In some embodiments, the first dose of the rhTNFR2-Fc-TPM fusion protein is administered 12 days before the observed platelet minimum in a chemotherapy immuno-oncology therapy cycle. In some embodiments, additional doses of the rhTNFR2-Fc-TPM fusion protein are administered weekly following the first dose. In some embodiments, additional doses of the rhTNFR2-Fc-TPM fusion protein are administered twice a week following the first dose. In some embodiments, additional doses of the rhTNFR2-Fc-TPM fusion protein are administered once every two weeks or at longer interval following the first dose. In some embodiments, the dosage for weekly administration versus twice a week administration versus once two weeks or three or four weeks or a series of administration are the same. In some embodiments, the dosage for weekly administration versus twice a week administration once two weeks or three or four weeks administration or a series of administration are different. In some embodiments, the drug is firstly administered in the first chemotherapy cycle. In some embodiments, the drug is firstly administered within 24 or 48 hours of the dose of chemotherapy immuno-oncology therapy in the second chemotherapy cycle. In some embodiments, the drug is firstly administered within 24 or 48 hours of the dose of chemotherapy immuno-oncology therapy in the third chemotherapy cycle.

In some aspects, administration of the fusion peptide or protein is determined by standard studies involving observation of platelet count or other symptoms in subjects. In some embodiments, the rhTNFR2-Fc-TPM fusion protein is administered when the platelet count of the patient is less than 400×10⁹, 300×10⁹, 200×10⁹, 1000×10⁹, 50×10⁹, 40×10⁹, 30×10⁹, 20×10⁹, or 10×10⁹/L.

In some embodiments, the rhTNFR2-Fc-TPM fusion protein is administered to patients that are refractory to other treatment methods. In some embodiment, the patients are refractory to TPIAO® (Recombinant Human Thrombopoietin, rHuTPO). In some embodiment, the patients are refractory to corticosteroids, immunoglobulins, or have insufficient response to splenectomy. In some embodiment, the patients are refractory to Enbrel® or etanercept. In some embodiment, the patients are refractory to Nplate® or romiplostim.

The rhTNFR2-Fc-TPM fusion protein described herein are provided to a subject in an amount effective to increasing megakaryocytes or platelets in the patient. The actual dosage of disclosed rhTNFR2-Fc-TPM fusion protein will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum therapeutic response.

In some embodiments, the rhTNFR2-Fc-TPM fusion protein is administered from 0.1 μg/kg to 100 mg/kg, for example, from about 1 μg/kg to about 50 mg/kg, such as about 1 μg/kg, about 2 μg/kg, about 3 μg/kg, about 6 μg/kg, about 8 μg/kg, about 10 μg/kg, about 13 μg/kg, about 15 μg/kg, about 20 μg/kg, about 25 μg/kg, about 30 μg/kg, about 40 μg/kg, about 50 μg/kg, about 100 μg/kg, about 200 μg/kg, about 300 μg/kg, about 600 μg/kg, about 800 μg/kg, about 1 mg/kg, about 1.2 mg/kg, about 1.5 mg/kg, about 2 mg/kg, about 3 mg/kg, about 5 mg/kg, about 10 mg/kg, about 20 mg/kg, about 30 mg/kg, or about 50 mg/kg.

The amount of rhTNFR2-Fc-TPM fusion protein described is determined based on the subject population (e.g., infant or elderly). An optimal amount for a particular composition can be ascertained by standard studies involving observation of serum concentration, platelet count, and other responses in subjects. It is understood that a therapeutically effective amount of the rhTNFR2-Fc-TPM fusion protein described herein, can be adjusted based on the observation of serum concentration, platelet count, and other responses in subjects, as well as other treatment, e.g. chemotherapy or immuno-oncology therapy, the subject received.

In some embodiments, decisions as to whether to change the amount of the therapeutic agent administered to the individual can be at least partially based on the platelet count. The platelet count can be based on, for example, a complete blood counts (CBCs), including platelet counts. The complete blood count may be performed each time before administration, weekly, or monthly. The amount of rhTNFR2-Fc-TPM fusion protein administered can be adjusted according to the complete blood counts and/or platelet counts results.

The platelet counts does not need to reach normal platelet counts for healthy individuals for the methods to be effective. For example, induce platelet generation by administering the rhTNFR2-Fc-TPM fusion protein described herein can increase platelet counts to a desired level, for example, higher than 10×10⁹, 20×10⁹, 30×10⁹, 40×10⁹, 50×10⁹, 100×10⁹, 200×10⁹, 300×10⁹, or 400×10⁹/L. In some embodiments, the rhTNFR2-Fc-TPM fusion protein described herein can increase platelet counts to a level sufficient to avoid clinically important bleeding.

In some embodiments, the rhTNFR2-Fc-TPM fusion protein can be administered to patients with cancer. In some embodiments, the cancer can be a liquid cancer such as blood cancers or malignancies of the lymphohematopoietic system. In some embodiments, the cancer is a solid cancer. In some embodiments, the cancer can be selected from the group consisting of a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, stomach cancer, thymic epithelial cancer, and thyroid cancer. In some embodiments, the cancer is in adjuvant setting. In some embodiments, the cancer can be in neoadjuvant setting. In some embodiments, the cancer can be advanced-stage cancer. In some embodiments, the cancer can be metastatic cancer.

In some embodiments, the cancer type can be a solid cancer type or a hematologic malignant cancer type. In some embodiments, the cancer type is a relapsed or refractory cancer type. In some embodiments, the cancer type can comprise acute myeloid leukemia (LAML or AML), acute lymphoblastic leukemia (ALL), adrenocortical carcinoma (ACC), bladder urothelial cancer (BLCA), brain stem glioma, brain lower grade glioma (LGG), brain tumor, breast cancer (BRCA), bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma (CESC) cancer, childhood cancers, cholangiocarcinoma (CHOL), chordoma, chronic lymphocytic leukemia, chronic myelogenous leukemia, chronic myeloproliferative disorders, colon (adenocarcinoma) cancer (COAD), colorectal cancer, craniopharyngioma, cutaneous T-cell lymphoma, endocrine pancreas islet cell tumors, endometrial cancer, ependymoblastoma, ependymoma, esophageal cancer (ESCA), esthesioneuroblastoma. Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioblastoma multiforme glioma GBM), hairy cell leukemia, head and neck cancer (HNSD), heart cancer, Hodgkin lymphoma, hypopharyngeal cancer, intraocular melanoma, islet cell tumors, Kaposi sarcoma, kidney cancer, Langerhans cell histiocytosis, laryngeal cancer, lip cancer, liver cancer, Lymphoid Neoplasm Diffuse Large B-cell Lymphoma (DLBCL), malignant fibrous histiocytoma bone cancer, medulloblastoma, medullo epithelioma, melanoma, Merkel cell carcinoma, Merkel cell skin carcinoma, mesothelioma (MESO), metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma (PCPG), pineal parenchymal tumors of intermediate differentiation, pineoblastoma, pituitary tumor, plasma cell neoplasm/multiple myeloma, pleuropulmonary blastoma, primary central nervous system (CNS) lymphoma, primary hepatocellular liver cancer, prostate cancer such as prostate adenocarcinoma (PRAD), rectal cancer, renal cancer, renal cell (kidney) cancer, renal cell cancer, respiratory tract cancer, retinoblastoma, rhabdomyosarcoma, salivary gland cancer, sarcoma (SARC), Sezary syndrome, skin cutaneous melanoma (SKCM), small cell lung cancer, small intestine cancer, soft tissue sarcoma, squamous cell carcinoma, squamous neck cancer, stomach (gastric) cancer, supratentorial primitive neuroectodermal tumors, T-cell lymphoma, testicular cancer testicular germ cell tumors (TGCT), throat cancer, thymic carcinoma, thymoma (THYM), thyroid cancer (THCA), transitional cell cancer, transitional cell cancer of the renal pelvis and ureter, trophoblastic tumor, ureter cancer, urethral cancer, uterine cancer, uterine cancer, uveal melanoma (UVM), vaginal cancer, vulvar cancer, Waldenstrom macroglobulinemia, or Wilm's tumor. In some embodiments, the cancer type can comprise acute lymphoblastic leukemia, acute myeloid leukemia, bladder cancer, breast cancer, brain cancer, cervical cancer, cholangiocarcinoma, colon cancer, colorectal cancer, endometrial cancer, esophageal cancer, gastrointestinal cancer, glioma, glioblastoma, head and neck cancer, kidney cancer, liver cancer, lung cancer, lymphoid neoplasia, melanoma, a myeloid neoplasia, ovarian cancer, pancreatic cancer, pheochromocytoma and paraganglioma, prostate cancer, rectal cancer, squamous cell carcinoma, testicular cancer, stomach cancer, or thyroid cancer.

In some embodiments, the rhTNFR2-Fc-TPM fusion protein can be administered via any suitable enteral route or parenteral route of administration. The term “enteral route” of administration refers to the administration via any part of the gastrointestinal tract. Examples of enteral routes include oral, sublingual, mucosal, buccal, and rectal route, or intragastric route. “Parenteral route” of administration refers to a route of administration other than enteral route. Examples of parenteral routes of administration include intravenous, intramuscular, intradermal, transdermal, intraperitoneal, intratumor, intravesical, intraarterial, intrathecal, intracapsular, intraorbital, intraosseous, intracardiac, transmucosal, transtracheal, intravitreal, intraarticular, peri-articular, subretinal, subcapsular, subarachnoid, intraspinal, epidural and intrasternal, subcutaneous, or topical administration.

In some embodiments, the rhTNFR2-Fc-TPM fusion protein can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art. In some embodiments, the TPO mimetic fusion protein and compositions of the disclosure is administered by subcutaneous injection. In some embodiments, the TPO mimetic fusion protein and compositions of the disclosure is administered by intravenous injection.

### VIII. Articles of Manufacture

Also provided are articles of manufacture or kits containing the provided recombinant polypeptide and proteins compositions. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., CIT or ITP). Alternatively. or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating CIT in an individual. The label or package insert may indicate that the composition is used for treating ITP in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating CIT or ITP an individual.

The container in some embodiments holds a composition which is by itself or combined with another composition effective for treating, preventing and/or diagnosing the condition. The article of manufacture or kit may include (a) a first container with a composition contained therein (first medicament), wherein the composition includes the fusion peptide or protein thereof; and (b) a second container with a composition contained therein (second medicament), wherein the composition includes a further agent, such as an another therapeutic agent, and which article or kit further comprises instructions on the label or package insert for treating the subject with the second medicament, in an effective amount.

### Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

“Fc region” as used herein refers to the polypeptide comprising the constant region of an antibody heavy chain excluding the first constant region immunoglobulin domain. For IgG, the Fc region may comprise immunoglobulin domains C_{H}2 and C_{H}3 and the hinge between C_{H}1 and C_{H}2. An Fc domain may have at least 80% sequence identity (e.g., at least 85%, 90%, 95%, 97%, or 100% sequence identity) to a human Fc domain that includes at least a C_{H}2 domain and a C_{H}3 domain. An Fc domain monomer includes second and third antibody constant domains (C_{H}2 and C_{H}3). In some embodiments, the Fc domain monomer also includes a hinge domain. An Fc domain does not include any portion of an immunoglobulin that is capable of acting as an antigen-recognition region, e.g., a variable domain or a complementarity determining region (CDR). In the wild-type Fc domain, the two Fc domain monomers dimerize by the interaction between the two C_{H}3 antibody constant domains, as well as one or more disulfide bonds that form between the hinge domains of the two dimerizing Fc domain monomers. In some embodiments, an Fc domain may be mutated to lack effector functions, typical of a “dead Fc domain.” In certain embodiments, each of the Fc domain monomers in an Fc domain includes amino acid substitutions in the C_{H}2 domain to reduce the interaction or binding between the Fc domain and an Fcγ receptor. In some embodiments, the Fc domain contains one or more amino acid substitutions that do not reduce or inhibit Fc domain dimerization. An Fc domain can be any immunoglobulin antibody isotype, including IgG, IgE, IgM, IgA, or IgD. Additionally, an Fc domain can be an IgG subtype (e.g., IgG1, IgG2a, IgG2b, IgG3, or IgG4). The Fc domain can also be a non-naturally occurring Fc domain, e.g., a recombinant Fc domain.

As used herein, “sequence identity” between two poly peptide sequences indicates the percentage of amino acids that are identical between the sequences. The amino acid sequence identity of polypeptides can be determined conventionally using known computer programs such as Bestfit, FASTA, or BLAST (see. e.g. Pearson, Methods Enzymol. 183:63-98 (1990); Pearson, Methods Mol Biol. 132185-219 (2000), Altschul et al. J. Mol. Biol. 215:403-410 (1990); Altschul et al., Nucleic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allow ed. This aforementioned method in determining the percentage of identity between polypeptides is applicable to all proteins, fragments, or variants thereof disclosed herein.

The term “host cell” refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NS0, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “isolated nucleic acid” refers to a nucleic acid molecule of genomic, cDNA, or synthetic origin, or a combination thereof, which is separated from other nucleic acid molecules present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (sequences located at the 5′ and 3′ ends of the nucleic acid of interest).

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

As used herein, the term “serum half-life” refers to, in the context of administering a therapeutic protein to a subject, the time required for plasma concentration of the protein in the subject to be reduced by half. The protein can be redistributed or cleared from the bloodstream, or degraded, e.g., by proteolysis.

Unless otherwise noted, a fusion protein is a recombinant protein containing amino acid sequence from at least two unrelated proteins that have been joined together, via a peptide bond, to make a single protein. The unrelated amino acid sequences can be joined directly to each other or they can be joined using a linker sequence. As used herein, proteins are unrelated, if their amino acid sequences are not normally found joined together via a peptide bond in their natural environment (e.g., inside a cell). For example, the amino acid sequences of a viral antigen and the amino acid sequences of a collagen or procollagen are not normally found joined together via a peptide bond.

Sequence identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Two sequences are “substantially identical” if two sequences have a specified percentage of amino acid residues or nucleotides that are the same (e.g., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### EXAMPLES

The following examples are included for illustrative purposes only and are not intended to limit the scope of the present disclosure.

### Example 1: rhTNFR2-Fc-TPM Construct, Expression, Purification, and Formulation

Construction of rhTNFR2-Fc-TPM Eukaryotic Expression Vector

The rhTNFR2-Fc-TPM expression vector contains both CMV and SV40 promotors, which drive the expression of the rhTNFR2-Fc-TPM fusion protein and a dihydrofolate reductase (DHFR), respectively. The DHFR function as selection marker for the expression vector and is used to select high expression cell lines through methotrexate (MTX) selection.

The coding region of the expression vector can be translated into a peptide (SEQ ID NO. 1) comprising a signal peptide (SEQ ID NO. 3), the TNFR2 region (SEQ ID NO. 4), the Fc region (SEQ ID NO. 5), and the functional peptide fragment repeats (SEQ ID NO. 6), from N-terminus to C-terminus.

Construction of Engineered rhTNFR2-Fc-TPM Cell Line

The rhTNFR2-Fc-TPM expression vector is stably transfected into the GH-CHO(dhfr^{-/-}) cells. Two highly expressed leading clones, 1F2B11 and 1F2E5, were selected through monoclonal screening under MTX selection.

### Expression and Formulation

The formulation is determined by IEF of the protein and accelerated test and high temperature test data. The final product is formulated as a sterile solution at pH 6.30±0.30 comprising rhTNFR2-Fc-TPM protein 1.00 g/L, sodium dihydrogen phosphate monohydrate 2.60 g/L, disodium hydrogen phosphate dihydrate 1.13 g/L, sodium chloride 5.80 g/L, sucrose 10.00 g/L, L-Arginine hydrochloride 5.30 g/L, polysorbate 20 0.042 g/L.

### Distribution of Bisulfide Bonds:

rhTNFR2-Fc-TPM is a dimeric glycoprotein with 16 pairs of disulfide bonds. Three pairs of the disulfide bonds are inter-polypeptide chain disulfide bonds: C240-C240, C246-C246, C249-249; thirteen pairs of the disulfide bonds are intra-polypeptide disulfide bonds: C18-C31, C32-C45, C35-C53, C56-C71, C78-C88, C78-C96, C98-C104, C112-C121, C115-C139, C142-C157, C163-C178, C281-C341, and C387-C445 (numbering based on SEQ ID NO: 2).

### N-Glycosylation Modification:

Through enzymatic digestion and chromatography analysis, two N-glycosylation modification sites. N149 and N171, were detected in rhTNFR2-Fc-TPM (numbering based on SEQ ID NO: 2), which matches the theoretical modification sites.

### Posttranslational Modification:

Through enzymatic digestion and chromatography analysis, two peptides containing oxidative modification sites were detected: M30 and M272; five peptides containing deamination modification sites were detected: Q82, Q109, N306, Q317, and Q524; one peptide containing aspartic acid isomerization modification sites were detected: D300 (numbering based on SEQ ID NO: 2).

### Example 2: Comparison with Romiplostim

BaF3/c-Mpl cell is a modified mouse B cell line expressing human TPOR. Its receptor can bind to human TPO and related substances with TPO activity, induce c-Mpl-JAK-STAT signal Tyrosine phosphorylation, activate cellular c-Mpl-JAK-STAT signaling pathway, and stimulate cell expansion. Therefore, BaF3/c-Mpl cell can grow in the presence of rhTNFR2-Fc-TPM, Romiplostim (Nplate®), or TPO in the absence of IL-3.

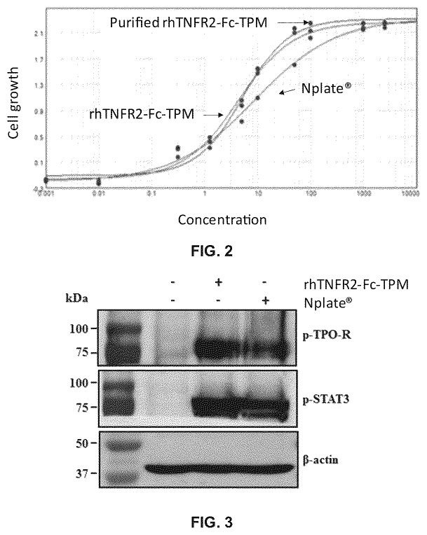

BaF3/c-Mpl cells were treated with different concentrations of rhTNFR2-Fc-TPM stock solution, formulated CB-219M, or Nplate®. The growth of BaF3/c-Mpl cells were analyzed by CCK-8 assay. As shown in Table 1, rhTNFR2-Fc-TPM had lower EC50. Additionally, the concentration curve of BaF3/c-Mpl cells treated with rhTNFR2-Fc-TPM had better uniformity and slope than BaF3/c-Mpl cells treated with Nplate® (FIG. 2), indicating the structure, receptor binding function, and quality uniformity of rhTNFR2-Fc-TPM is better than that of Nplate®.

TABLE 1
Comparison of the bioactivity of rhTNFR2-Fc-TPM and Nplate ® by
CCK-8 assay in BaF3/c-Mpl cells.

| | rhTNFR2-Fc-TPM | Formulated | |
|---|---|---|---|
| | Stock Solution | rhTNFR2-Fc-TPM | Nplate ® |
| EC₅₀ (ng/ml) | 4.261 | 5.161 | 8.775 |

### Example 3: In Vitro Efficacy Study

Study of the Effects of rhTNFR2-Fc-TPM on c-Mpl-JAK-STA T Signaling Pathway in BaF3/c-Mpl Cells

BaF3/c-Mpl cells were treated with 0.5 mg/ml rhTNFR2-Fc-TPM or 0.5 mg/ml Nplate®. After treatment, the cells were collected by centrifugation and lysed by ultra sound. The samples were analyzed by Western-blot using phosphor-specific antibodies.

As shown in FIG. 3, phosphorylated TPOR and phosphorylated STAT3 were detected both in cells treated with rhTNFR2-Fc-TPM and cells treated with Nplate® demonstrating both rhTNFR2-Fc-TPM and Nplate® can activate TPOR-JAK-STAT3 signaling pathway. These results indicate that rhTNFR2-Fc-TPM and Nplate® have the same mechanism of action.

### Example 4: In Vitro Efficacy Study

Study of the Effect of rhTNFR2-Fc-TPM on Healthy Balb/c Mice Platelet Generation

Six to eight week old female Balb/c mice were treated with a single dose of rhTNFR2-Fc-TPM through different route of administration. Nplate® was used as a positive control and PBS solution was used as a negative control. The details of the study design is shown in Table 2.

TABLE 2
Design of administration routes and dosage studies
of rhTNFR2-Fc-TPM in healthy Balb/c mice

| | | | | | Volume | Number of |
|---|---|---|---|---|---|---|
| Group | | Route of | Dosage | Concentration | Administered | Animals |
| Number | Drug | Administration | (μg/kg) | (μg/ml) | (μl/ea) | in Group |
| 1 | PBS solution | — | — | — | 100 | 5 |
| | (Negative | | | | | |
| | control) | | | | | |
| 2 | Nplate ® | Subcutaneous | 50 | 11 | 100 | 5 |
| | | injection | | | | |
| 3 | rhTNFR2- | Subcutaneous | 5 | 1.1 | 100 | 5 |
| | Fc-TPM | injection | | | | |
| 4 | rhTNFR2- | Subcutaneous | 10 | 2.2 | 100 | 5 |
| | Fc-TPM | injection | | | | |
| 5 | rhTNFR2- | Subcutaneous | 50 | 11 | 100 | 5 |
| | Fc-TPM | injection | | | | |
| 6 | rhTNFR2- | Subcutaneous | 100 | 22 | 100 | 5 |
| | Fc-TPM | injection | | | | |
| 7 | rhTNFR2- | Subcutaneous | 200 | 44 | 100 | 5 |
| | Fc-TPM | injection | | | | |
| 8 | Nplate ® | Intravenous | 50 | 11 | 100 | 5 |
| | | injection | | | | |
| 9 | rhTNFR2- | Intravenous | 5 | 1.1 | 100 | 5 |
| | Fc-TPM | injection | | | | |
| 10 | rhTNFR2- | Intravenous | 10 | 2.2 | 100 | 5 |
| | Fc-TPM | injection | | | | |
| 11 | rhTNFR2- | Intravenous | 50 | 11 | 100 | 5 |
| | Fc-TPM | injection | | | | |
| 12 | rhTNFR2- | Intravenous | 100 | 22 | 100 | 5 |
| | Fc-TPM | injection | | | | |
| 13 | rhTNFR2- | Intravenous | 200 | 44 | 100 | 5 |
| | Fc-TPM | injection | | | | |

The mice in each group were weighted and blood samples were taken from the orbital venous plexus at day −1, 3, 4, 5, 6, 7, 9, and 11. The body weight and platelet count were analyzed by GraphPad Prism 7 software using Two-way ANOVA analysis method.

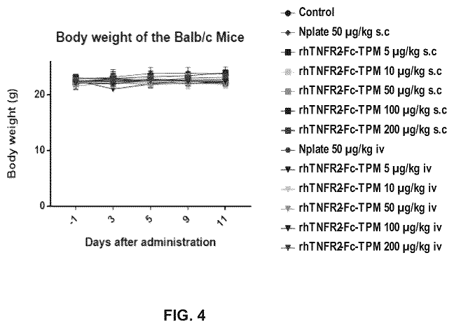

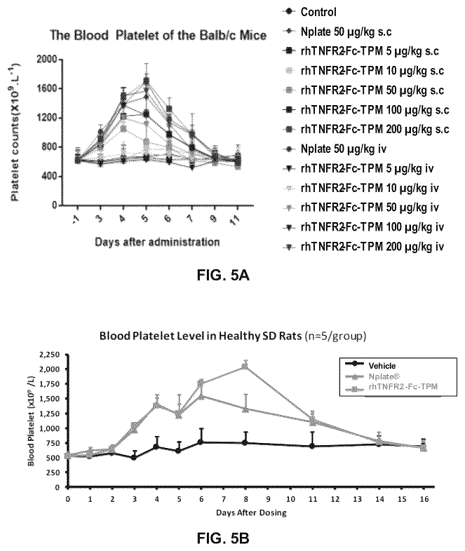

As shown in FIG. 4, the body weight of the mice in each group did not change significantly; the body weight did not differ significantly on day 0 and day 11 among all treatment groups (p>0.05). As shown in FIG. 5A, treatment with rhTNFR2-Fc-TPM at all three dosage levels (50, 100, and 200 μg/kg) increased platelet counts in female Balb/c mice and there was no significant differences between administration routes. The increase of platelet counts positively correlated with rhTNFR2-Fc-TPM dosage and the higher dosages showed delayed platelet count peak compared to lower dosages; the platelet counts of each treatment groups returned to normal level 9-11 days after administration (FIG. 5A). Similar results were observed in healthy SD rats treated with rhTNFR2-Fc-TPM (FIG. 5B)

Study of the Effects of rhTNFR2-Fc-TPM on Carboplatin Induced CIT Mice Model

The CIT mice model was produced by intraperitoneal injection of 125 mg/kg carboplatin in the mice before drug administration. The details of the study design is shown in Table 3.

TABLE 3
Design of dosage studies of rhTNFR2-Fc-TPM in CIT mice model

| | | | | | Volume | Number of |
|---|---|---|---|---|---|---|
| Group | | Route of | Dosage | Concentration | Administered | Animals |
| Number | Drug | Administration | (μg/kg) | (μg/ml) | (μl/ea) | in Group |
| 1 | PBS solution | Subcutaneous | — | — | 100 | 7 |
| | (Negative | injection | | | | |
| | control) | | | | | |
| 2 | Nplate ® | Subcutaneous | 100 | 22 | 100 | 7 |
| | | injection | | | | |
| 3 | rhTNFR2- | Subcutaneous | 100 | 22 | 100 | 7 |
| | Fc-TPM | injection | | | | |
| 4 | rhTNFR2- | Subcutaneous | 200 | 44 | 100 | 7 |
| | Fc-TPM | injection | | | | |
| 5 | rhTNFR2- | Subcutaneous | 400 | 88 | 100 | 7 |
| | Fc-TPM | injection | | | | |

The mice in each group were weighted and blood samples were taken from the orbital venous plexus at day −1, 3, 5, 8, 11, 14, 16, 19 and 22. The body weight and platelet count were analyzed by GraphPad Prism 7 software using Two-way ANOVA analysis method.

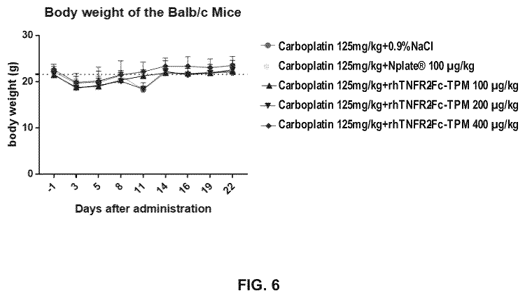

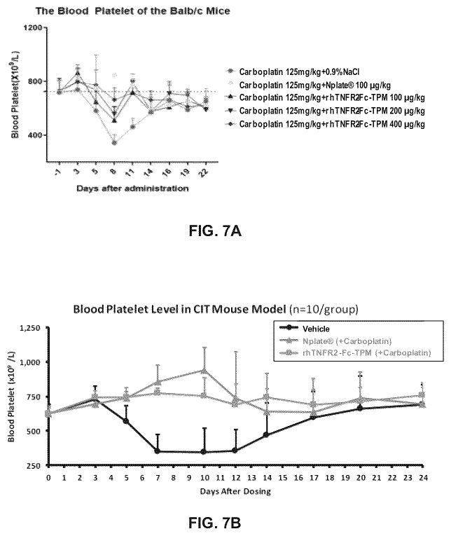

As shown in FIG. 6, no abnormality of body weight was observed in any treatment group; the average body weight of each treatment grope did not differ significantly (p>0.05). As shown in FIG. 7A, at all three dosage levels (100, 200, and 400 μg/kg) of rhTNFR2-Fc-TPM showed good treatment effects in carboplatin induced CIT in mice. The increase of platelet counts positively correlated with rhTNFR2-Fc-TPM dosage (FIG. 7A). Similar results were observed in carboplatin induced CIT SD rats treated with rhTNFR2-Fc-TPM (FIG. 7B)

The in vivo effective studies showed that a single dose of rhTNFR2-Fc-TPM administered subcutaneously can induce platelet count increase in mice, the effective dose is >10 μg/kg. rhTNFR2-Fc-TPM showed good efficacy in carboplatin induced CIT model and the increase of platelet count positively correlated with dosage. rhTNFR2-Fc-TPM is well tolerated and it efficacy did not show significant differences between administration routes.

### Example 5: In Vivo Pharmacodynamics Study

SD rats were administrated with a single dose of rhTNFR2-Fc-TPM subcutaneously at 0.02 mg/kg (low dose), 0.06 mg/kg (medium dose), or 0.2 mg/kg (high dose) or administered intravenously at 0.2 mg/kg. For subcutaneous administration, blood samples were taken before administration, and at 2, 8, 12, 14, 24 hours and 2, 3, 4, 5, 7 days after administration; for intravenous administration, blood samples were taken at 3 mi, 2, 8, 12, 14, 24 hours, and 2, 3, 4, 5, 7 days. Cynomolgus monkeys were administrated with a single dose of rhTNFR2-Fc-TPM subcutaneously at 0.02 mg/kg (low dose), 0.06 mg/kg (medium dose), or 0.2 mg/kg (high dose) or intravenously at 0.2 mg/kg. For subcutaneous administration, blood samples were taken before administration and at 2, 8, 12, 24 hours, 2, 3, 6, 8, 11, 15 days after administration; for intravenous administration, blood samples were taken at 5 m, 2, 8, 12, 24 hours, and 2, 3, 6, 8, 11, 15 days. rhTNFR2-Fc-TPM concentration in the serum were analyzed using ELISA method, the pharmacodynamics were analyzed by WinNonlin 8.0 using non-compartmental model.

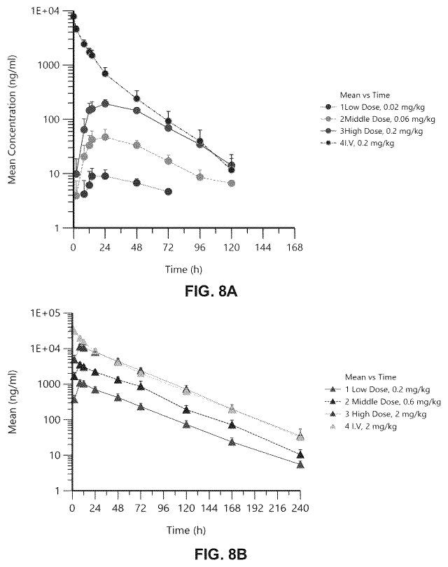

As shown in FIGS. 8A-8B, the serum drug concentrations positively correlated with the dosage administered. The trends of serum drug concentration changes were same in male and female animals tested (data not shown). The half-life in SD rats was between 22.2-32.7 hours (FIG. 8A); the half-life in cynomolgus monkeys was between 26.9-29.5 hours (FIG. 89) drug serum half-life was not related to dose administered in either SD rats or cynomolgus monkeys (FIGS. 8A-8B). Pharmacodynamics analysis showed that AUC and Cₘₐₓ in both SD rats and cynomolgus monkeys increased with increase of drug dosage (Table 4 and Table 5)

TABLE 4
Pharmacodynamics parameters (Mean ± SD) of single dose of rhTNFR2-Fc-TPM in SD rats.

| Group | | Tₘₐₓ | t_{1/2} | Cₘₐₓ | AUC_{0-168 h} | AUCₗₐₛₜ | AUC_{inf} | Vd | Cl | MRTₗₐₛₜ |
|---|---|---|---|---|---|---|---|---|---|---|
| (mg/kg) | Sex | (h) | (h) | (μg/mL) | (h · μg/mL) | (h · μg/mL) | (h · μg/mL) | (mL/kg) | (mL/h/kg) | (h) |
| Low dose | M | 36.7 ± | 38.4 ± | 8.36 ± | 0.271 ± | 0.372 ± | 0.716 ± | 1530 ± | 28.2 ± | 36.3 ± |
| 0.02 mg/kg | | 19.6 | 11.5 | 1.57 | 0.0800 | 0.157 | 0.0969 | 258 | 3.82 | 4.71 |
| | F | 17.3 ± | 29.0 ± | 12.2 ± | 0.385 ± | 0.385 ± | 0.637 ± | 1340 ± | 32.0 ± | 24.5 ± |
| | | 5.77 | 2.89 | 0.666 | 0.0624 | 0.0624 | 0.106 | 298 | 5.80 | 0.810 |
| | Total | 27.0 ± | 32.7 ± | 10.3 ± | 0.328 ± | 0.378 ± | 0.668 ± | 1420 ± | 30.5 ± | 30.4 ± |
| | | 16.7 | 8.00 | 2.38 | 0.0896 | 0.107 | 0.0992 | 268 | 4.99 | 7.15 |
| Medium dose | M | 24.0 ± | 25.4 ± | 36.2 ± | 1.34 ± | 2.37 ± | 2.64 ± | 855 ± | 23.3 ± | 44.6 ± |
| 0.06 mg/kg | | 0.00 | 0.266 | 3.18 | 0.179 | 0.552 | 0.541 | 153 | 4.39 | 4.30 |
| | F | 24.0 ± | 21.3 ± | 58.2 ± | 2.00 ± | 2.82 ± | 3.06 ± | 711 ± | 21.9 ± | 35.6 ± |
| | | 0.00 | 4.59 | 21.6 | 0.808 | 1.18 | 1.08 | 469 | 9.71 | 0.606 |
| | Total | 24.0 ± | 23.3 ± | 47.2 ± | 1.67 ± | 2.59 ± | 2.85 ± | 783 ± | 22.6 ± | 40.1 ± |
| | | 0.00 | 3.68 | 18.3 | 0.636 | 0.857 | 0.801 | 322 | 6.79 | 5.65 |
| High dose | M | 24.0 ± | 25.1 ± | 175 ± | 5.84 ± | 10.5 ± | 11.1 ± | 675 ± | 18.4 ± | 47.1 ± |
| 0.2 mg/kg | | 0.00 | 3.75 | 28.6 | 0.968 | 1.87 | 1.78 | 223 | 3.23 | 1.02 |
| | F | 20.0 ± | 19.3 ± | 213 ± | 7.68 ± | 11.9 ± | 12.2 ± | 459 ± | 16.7 ± | 40.5 ± |
| | | 6.93 | 2.41 | 38.6 | 1.15 | 1.78 | 1.94 | 40.9 | 2.45 | 1.61 |
| | Total | 22.0 ± | 22.2 ± | 194 ± | 6.76 ± | 11.2 ± | 11.6 ± | 567 ± | 17.5 ± | 43.8 ± |
| | | 4.90 | 4.25 | 36.8 | 1.39 | 1.80 | 1.77 | 186 | 2.72 | 3.81 |
| I.V. | M | 0.0500 ± | 16.1 ± | 7460 ± | 74.7 ± | 82.9 ± | 83.3 ± | 55.3 ± | 2.43 ± | 17.1 ± |
| 0.2 mg/kg | | 0.00 | 3.50 | 501 | 8.03 | 11.0 | 11.4 | 5.47 | 0.345 | 1.97 |
| | F | 0.050 ± | 15.6 ± | 8110 ± | 58.5 ± | 62.7 ± | 62.9 ± | 73.2 ± | 3.23 ± | 13.7 ± |
| | | 0.00 | 1.11 | 894 | 8.76 | 9.59 | 9.59 | 16.2 | 0.506 | 0.690 |
| | Total | 0.050 ± | 15.8 ± | 7780 ± | 66.6 ± | 72.8 ± | 73.1 ± | 64.2 ± | 2.83 ± | 15.4 ± |
| | | 0.00 | 2.34 | 738 | 11.6 | 14.4 | 14.6 | 14.6 | 0.585 | 2.33 |

TABLE 5
Pharmacodynamics parameters (Mean ± SD) of single dose of rhTNFR2-Fc-TPM in cynomolgus monkeys.

| Group | | Tₘₐₓ | t_{1/2} | Cₘₐₓ | AUC_{0-168 h} | AUCₗₐₛₜ | AUC_{inf} | Vd | Cl | MRTₗₐₛₜ |
|---|---|---|---|---|---|---|---|---|---|---|
| (mg/kg) | Sex | (h) | (h) | (μg/mL) | (h · μg/mL) | (h · μg/mL) | (h · μg/mL) | (mL/kg) | (mL/h/kg) | (h) |
| Low dose | M | 8.00 ± | 29.0 ± | 1.01 ± | 45.5 ± | 45.9 ± | 46.5 ± | 195 ± | 4.67 ± | 41.7 ± |
| 0.02 mg/kg | | 0.00 | 0.691 | 0.322 | 14.2 | 14.7 | 14.7 | 70.6 | 1.76 | 3.08 |
| | F | 9.33 ± | 29.9 ± | 1.17 ± | 55.2 ± | 56.0 ± | 56.5 ± | 153 ± | 3.55 ± | 42.8 ± |
| | | 2.31 | 1.32 | 0.0757 | 3.35 | 4.02 | 3.74 | 3.37 | 0.231 | 3.41 |
| | Total | 8.67 ± | 29.5 ± | 1.09 ± | 50.3 ± | 50.9 ± | 51.5 ± | 174 ± | 4.11 ± | 42.3 ± |
| | | 1.63 | 1.06 | 0.225 | 10.7 | 11.1 | 11.0 | 50.3 | 1.28 | 2.96 |
| Medium dose | M | 8.00 ± | 28.0 ± | 3.35 ± | 146 ± | 149 ± | 149 ± | 164 ± | 4.06 ± | 40.7 ± |
| 0.06 mg/kg | | 0.00 | 0.914 | 0.541 | 14.5 | 14.7 | 14.7 | 20.1 | 0.417 | 1.72 |
| | F | 8.00 ± | 27.4 ± | 3.65 ± | 178 ± | 180 ± | 181 ± | 134 ± | 3.40 ± | 43.3 ± |
| | | 0.00 | 1.19 | 0.580 | 33.6 | 34.4 | 35.0 | 26.3 | 0.715 | 4.57 |
| | Total | 8.00 ± | 27.7 ± | 3.50 ± | 162 ± | 164 ± | 165 ± | 149 ± | 3.73 ± | 42.0 ± |
| | | 0.00 | 1.01 | 0.528 | 29.0 | 29.2 | 29.9 | 26.6 | 0.636 | 3.41 |
| High dose | M | 8.00 ± | 27.4 ± | 11.0 ± | 535 ± | 544 ± | 546 ± | 146 ± | 3.69 ± | 42.6 ± |
| 0.2 mg/kg | | 0.00 | 2.50 | 0.520 | 57.6 | 60.1 | 60.8 | 16.8 | 0.413 | 2.77 |
| | F | 8.00 ± | 26.4 ± | 11.6 ± | 534 ± | 542 ± | 543 ± | 149 ± | 3.94 ± | 40.8 ± |
| | | 0.00 | 1.74 | 2.79 | 159 | 163 | 163 | 47.2 | 1.28 | 0.563 |
| | Total | 8.00 ± | 26.9 ± | 11.3 ± | 535 ± | 543 ± | 545 ± | 147 ± | 3.81 ± | 41.7 ± |
| | | 0.00 | 2.00 | 1.82 | 107 | 110 | 110 | 31.8 | 0.862 | 2.06 |
| I.V. | M | 0.0800 ± | 29.5 ± | 40.7 ± | 726 ± | 732 ± | 735 ± | 116 ± | 2.72 ± | 28.8 ± |
| 0.2 mg/kg | | 0.00 | 0.875 | 1.39 | 11.9 | 16.1 | 14.1 | 2.06 | 0.0516 | 1.17 |
| | F | 0.0800 ± | 26.6 ± | 36.8 ± | 766 ± | 771 ± | 774 ± | 102 ± | 2.63 ± | 28.6 ± |
| | | 0.00 | 2.72 | 9.45 | 134 | 138 | 136 | 25.0 | 0.419 | 0.643 |
| | Total | 0.0800 ± | 28.1 ± | 38.8 ± | 746 ± | 751 ± | 755 ± | 109 ± | 2.68 ± | 28.7 ± |
| | | 0.00 | 2.41 | 6.41 | 88.1 | 90.6 | 89.0 | 17.6 | 0.271 | 0.848 |

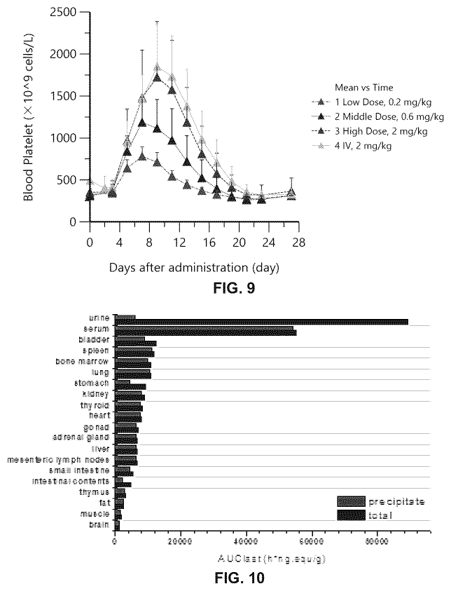

As shown in FIG. 9, all dosage levels and administration routes induced significant increase of platelet count in cynomolgus monkeys and the level of increase positively correlated with the dosage administered.

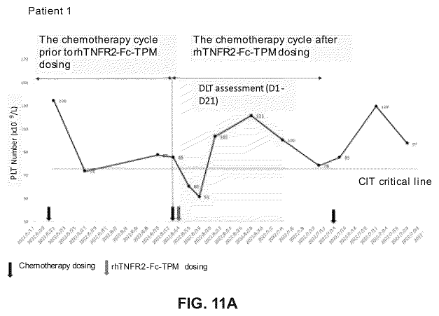

Study of single dose of ¹²⁵I labeled rhTNFR2-Fc-TPM shows the highest distribution of rhTNFR2-Fc-TPM was in serum and rhTNFR2-Fc-TPM was mainly excreted through urine (FIG. 10). 94.91±1.53% of ¹²⁵I labeling was excreted through urine and stool 12 days after administration, accumulation of rhTNFR2-Fc-TPM in the body was not observed (data not shown).

### Example 6: In Vivo Safety and Toxicology Study

### Safety Study

SD rats administered a single dose of rhTNFR2-Fc-TPM subcutaneously at 50, 200, or 1000 μg/kg did not show significant change of the function of the central nervous system. No significant changes in ECG parameters and waveform, respiratory rate and tidal volume, or blood pressure related to rhTNFR2-Fc-TPM administration (p>0.05) was observed in cynomolgus monkeys administrated rhTNFR2-Fc-TPM twice a week for 4 weeks at 300, 1000, 3000 μg/kg.

### Acute Toxicology Study

SD rats were administered a single dose of rhTNFR2-Fc-TPM subcutaneously at 100, 300, or 1000 μg/kg; cynomolgus monkeys were administered a single dose of rhTNFR2-Fc-TPM subcutaneously at 500, 1500, or 5000 μg/kg. The animals were observed for two weeks after rhTNFR2-Fc-TPM administration. No significant change in body weight, body temperature. ECG, or clinical pathology (hematology, blood biochemistry, blood coagulation, and urinalysis). The animals were sacrificed and dissected 15 days after rhTNFR2-Fc-TPM administration. No significant abnormal pathological changes were observed.

### Long-Term Toxicology Study

rhTNFR2-Fc-TPM was administered twice a week for 4 weeks in SD rats at 20, 60, 200 μg/kg and in cynomolgus monkeys at 300, 1000, 3000 μg/kg, followed by a 4-week recovery period.

During treatment period. SD rats treated with ≥20 μg/kg rhTNFR2-Fc-TPM showed increase of platelet, IL-2, IL-6, and TNF-α and decrease of hemoglobin. Additionally, SD rats treated with ≥60 μg/kg rhTNFR2-Fc-TPM showed increase of white blood cells, neutrophils, lymphocytes, monocytes, and basophils. These changes were recovered during the recovery period. No abnormality related to drug administration was observed in clinical observation, weight, food intake, body temperature, eye examination, urine examination, coagulation function, blood biochemistry, T lymphocyte subsets, gross anatomy, or histopathology. The no-observed-adverse-effect level (NOAEL) for SD rats was 200 μg/kg.

During treatment period, cynomolgus monkeys treated with 300, 1000, and 3000 μg/kg rhTNFR2-Fc-TPM showed slight to mild dermal and/or subcutaneous inflammatory cell infiltration; increase of platelet, and K+ levels; and decrease of red blood cell count, hemoglobin, hematocrit, and mean corpuscular hemoglobin concentration. Additionally, cynomolgus monkeys treated with 1000, and 3000 μg/kg rhTNFR2-Fc-TPM showed increase of reticulocytes count. These changes were recovered during the recovery period. No abnormality related to drug administration was observed in clinical observation, weight, food intake, body temperature, eye examination, urine examination, coagulation function, lymphocyte subsets, gross anatomy, or histopathology. The NOAEL for cynomolgus monkeys was 3000 μg/kg.

The AUC and Cₘₐₓ in both SD rats and cynomolgus monkeys positively correlated with the dose administered; no accumulation of rhTNFR2-Fc-TPM was observed in SD rats or cynomolgus monkeys.

### In Vitro Hemolysis Study

2% red blood cells saline suspension was incubated with 0.1-0.5 ml formulated rhTNFR2-Fc-TPM (contained 2.00 mg/mL rhTNFR2-Fc-TPM) at 37° C. for 3 hours. No hemolysis or coagulation of red blood cell was observed.

### Example 7: Phase I Clinical Study

The Phase I clinical study will consists of two phases: dose escalation phase and dose expansion phase. A single dose will be administered to each patient in the dose escalation phase of the study; multiple doses will be administered to each patient in the dose expansion phase of the study.

### Dose Escalation Phase

The main goals for the dose escalation phase of the study will be: to evaluate the safety, tolerability, and immunogenicity of the single ascending dose (SAD) of rhTNFR2-Fc-TPM when administered subcutaneously; and to explore the maximum tolerated dose (MTD) and biologically effective dose (BED) of rhTNFR2-Fc-TPM.

The secondary goals for the dose escalation phase of the study will be: to evaluate pharmacodynamics (PK) characteristics of rhTNFR2-Fc-TPM in serum after a single dose; and to evaluate the changes of platelet count during chemotherapy cycle after a single dose of rhTNFR2-Fc-TPM.

Based on efficacy, pharmacodynamics, safety, toxicology studies in Balb/c mice, SD rats, and cynomolgus monkeys, the starting dose of the dose escalation phase will be 2 μg/kg, the maximum dose will be 15 μg/kg. Enrolled patients will be divided in to 4 cohorts: 2, 6, 10, and 15 μg/kg. Each patient will receive a single subcutaneous abdominal injection of rhTNFR2-Fc-TPM at the dosage of their cohort 6-24 hours after the first dose of their first chemotherapy cycle.

### Dose Expansion Phase

The main goals for the dose expansion phase of the study will be: to evaluate the safety, tolerability, and immunogenicity of the MTD or BED dose of rhTNFR2-Fc-TPM when administered subcutaneously once a week.

The secondary goals for the dose expansion phase of the study phase will be: to evaluate pharmacodynamics (PK) characteristics of rhTNFR2-Fc-TPM in serum when administered subcutaneously once a week; to compare the safety and preliminary efficacy of twice-weekly versus once-weekly subcutaneous rhTNFR2-Fc-TPM administration; to compare the preliminary efficacy of twice-weekly versus once-weekly administration of rhTNFR2-Fc-TPM for the management of grade 3 or grade 4 CIT; to evaluate the preliminary efficacy of using rhTNFR2-Fc-TPM to prevent grade 3 or grade 4 thrombocytopenia based on the last cycle's platelet minimum.

Enrolled patents will be divided in to 3 cohorts:

### Cohort A:

In the first cycle (C1), rhTNFR2-Fc-TPM will be administered by abdominal subcutaneous injection 6-24 h after the first day of the first chemotherapy cycle (C1D1). The starting dose will be the BED determined in the dose escalation phase, followed by weekly dosing (subsequent doses will be adjusted according to platelet counts), the subsequent dosing days in this cycle will include C1D8 and D15.

In the second cycle (C2), no drug will be administered. The platelet minimum for the cycle will be assessed and the date of the platelet minimum in C2 will be recorded as Dmin (e.g., D14).

In the third cycle (C3), if Dmin≤D12, the drug will be administered after chemotherapy at D1; if Dmin>12, the drug will be administered at Dmin−12 (if Dmin is 18, the drug is administered at C3D6 (after chemotherapy, if applicable)); the first time of drug administration in C3 will be recorded as day Dx; the drug will be administered every 7 days thereafter (Dx+7), up to the completion of the cycle (excluding C3D21).

### Cohort B:

No drug will be administered in the first cycle (C1).

In the second cycle (C2), rhTNFR2-Fc-TPM will be administered by abdominal subcutaneous injection 6-24 h after the first day of the second chemotherapy cycle (C2D1). The starting dose will be the BED determined in the dose escalation phase, followed by weekly dosing (subsequent doses will be adjusted according to platelet counts), the subsequent dosing days in this cycle will include C2D8 and D15.

The third cycle dosing schedule will be the same as the third cycle for Group A.

### Group C:

In the first cycle (C1), rhTNFR2-Fc-TPM will be administered by abdominal subcutaneous injection 6-24 h after the first day of the first chemotherapy cycle (C1D1). The starting dose will be the BED determined in the dose escalation phase, followed by twice weekly dosing (subsequent doses adjusted according to PLT counts), with subsequent dosing dates in this cycle including C1D4/D8/D11/D15/D18).

No drug will be administered in the second cycle (C2).

In the third cycle (C3), the first dose of C3 will be given on Dx (see determination of Dx in C3 of cohort A); subsequent doses will be given every 3 days (Dx+3), up to the completion of the cycle (excluding C3D21).

### Example 8: Pharmacodynamics Studies in a Phase I Clinical Study

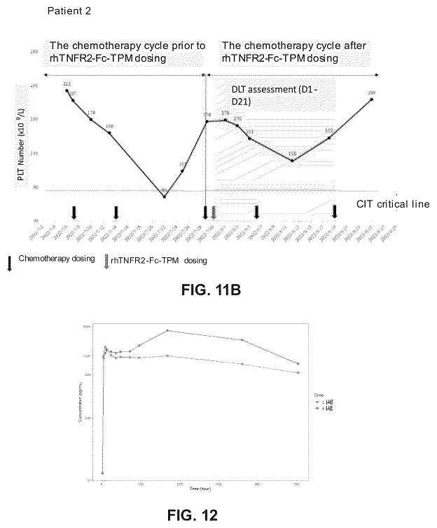

FIGS. 11A and 11B show pharmacodynamics curves of rhTNFR2-Fc-TPM in serum of patient 1 and patient 2, who had CIT caused by chemotherapy, after administrations of a single dose of rhTNFR2-Fc-TPM. 2 μg/Kg of rhTNFR2-Fc-TPM was administrated to patient 1 and 6 μg/Kg of rhTNFR2-Fc-TPM was administrated to patient 2 by abdominal subcutaneous injection.

It was observed in FIGS. 11A and 11B that in the chemotherapy cycle prior to the administration of rhTNFR2-Fc-TPM, the PLT numbers in both patients dropped below the CIT critical line (PLT=75). It was also observed that the rhTNFR2-Fc-TPM administrated to the patients was able to increase the PLT numbers in the subsequent two chemotherapy cycles.

The Dose Limiting Toxicity (DLT) was assessed from D1 to D21 following the administration of the drug candidate and both low and medium doses show a favorable safety profiling in terms of adverse reactions, e.g., fever, chills, general discomfort, fatigue, knee pain, headache, dizziness, and blood pressure.

### Example 9: Pharmacokinetic Studies in a Phase I Clinical Study

FIG. 12 shows pharmacokinetics curves of rhTNFR2-Fc-TPM in serum after administrations of a single dose of rhTNFR2-Fc-TPM to patient 1 and patient 2, respectively.

Consistent with the pharmacodynamics studies discussed in Example 8, the plasma concentration (Cmax) is proportional to the administered dose, and the half-life (T_{1/2}) is about 2 weeks. Patient 1 given the drug at 2 μg/Kg and the resulted Cmax is 1390 μg/mL, t_{1/2} is 368 hour (15.3 days) while patient 2 given the drug at 6 μg/Kg and the resulted Cmax is 2530 μg/mL and t_{1/2} is ˜300 hour (12.5 days). Thus, the dosing frequency of once every 2 weeks or longer intervals (e.g., once every 2.5, 3, 3.5, 4, 4.5 weeks) is supported by the fact that the first two CIT patients having a single dose of rhTNFR2-Fc-TPM demonstrated platelet maintenance on subsequent two chemotherapy cycles or longer and with the clear trend that the plasma concentration (Cmax) is proportional to the administered dose. As a result, compared with existing CIT therapeutic drugs, e.g., TPIAO® (rHuTPO)) requiring daily visit for 14 days, rhTNFR2-Fc-TPM, requiring much fewer hospital visits, will bring significant advantages to patient treatment and economic burden with less patients' suffering and economic costs.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID NO. | SEQUENCE | DESCRIPTION |
|---|---|---|
| 1 | MAPVAVWAALAVGLELWAAALPAQVAFTPYAPEPGSTCRLREYYDQTAQM | TNFR2-TPO |
| | CCSKCSPGQHAKVFCTKTSDTVCDSCEDSTYTQLWNWVPECLSCGSRCSS | mimetic fusion |
| | DQVETQACTREQNRICTCRPGWYCALSKQEGCRLCAPLRKCRPGFGVARP | polypeptide |
| | GTETSDVVCKPCAPGTFSNTTSSTDICRPHQICNVVAIPGNASMDAVCTS | amino acid |
| | TSPTRSMAPGAVHLPQPVSTRSQHTQPTPEPSTAPSTSFLLPMGPSPPAE | sequence (with |
| | GSTGDEPKSCDKTHTCPPCPAPELLGGPSVFLFPPKPKDTLMISRTPEVT | signal |
| | CVVVDVSHEDPEVKFNWYVDGVEVHNAKTKPREEQYQSTYRVVSVLTVLH | peptide) |
| | QDWLNGKEYKCKVSNKALPAPIEKTISKAKGQPREPQVYTLPPSRDELTK | |
| | NQVSLTCLVKGFYPSDIAVEWESNGQPENNYKTTPPVLDSDGSFFLYSKL | |
| | TVDKSRWQQGNVFSCSVMHEALHNHYTQKSLSLSPGKGGGGGIEGPTLRQ | |
| | WLAARAGGGGGGGGIEGPTLRQWLAARAGGGGGGGGIEGPTLRQWLAARA | |
| 2 | LPAQVAFTPYAPEPGSTCRLREYYDQTAQMCCSKCSPGQHAKVFCTKTSD | TNFR2-TPO |
| | TVCDSCEDSTYTQLWNWVPECLSCGSRCSSDQVETQACTREQNRICTCRP | mimetic fusion |
| | GWYCALSKQEGCRLCAPLRKCRPGFGVARPGTETSDVVCKPCAPGTFSNT | polypeptide |
| | TSSTDICRPHQICNVVAIPGNASMDAVCTSTSPTRSMAPGAVHLPQPVST | amino acid |
| | RSQHTQPTPEPSTAPSTSFLLPMGPSPPAEGSTGDEPKSCDKTHTCPPCP | sequence |
| | APELLGGPSVFLFPPKPKDTLMISRTPEVTCVVVDVSHEDPEVKFNWYVD | (without |
| | GVEVHNAKTKPREEQYQSTYRVVSVLTVLHQDWLNGKEYKCKVSNKALPA | signal |
| | PIEKTISKAKGQPREPQVYTLPPSRDELTKNQVSLTCLVKGFYPSDIAVE | peptide) |
| | WESNGQPENNYKTTPPVLDSDGSFFLYSKLTVDKSRWQQGNVFSCSVMHE | |
| | ALHNHYTQKSLSLSPGKGGGGGIEGPTLRQWLAARAGGGGGGGGIEGPTL | |
| | RQWLAARAGGGGGGGGIEGPTLRQWLAARA | |
| 3 | MAPVAVWAALAVGLELWAAA | Signal peptide |
| 4 | LPAQVAFTPYAPEPGSTCRLREYYDQTAQMCCSKCSPGQHAKVFCTKTSD | TNFR2 region |
| | TVCDSCEDSTYTQLWNWVPECLSCGSRCSSDQVETQACTREQNRICTCRP | amino acid |
| | GWYCALSKQEGCRLCAPLRKCRPGFGVARPGTETSDVVCKPCAPGTFSNT | sequence |
| | TSSTDICRPHQICNVVAIPGNASMDAVCTSTSPTRSMAPGAVHLPQPVST | |
| | RSQHTQPTPEPSTAPSTSFLLPMGPSPPAEGSTGD | |
| 5 | EPKSCDKTHTCPPCPAPELLGGPSVFLFPPKPKDTLMISRTPEVTCVVVD | Fc region |
| | VSHEDPEVKFNWYVDGVEVHNAKTKPREEQYQSTYRVVSVLTVLHQDWLN | amino acid |
| | GKEYKCKVSNKALPAPIEKTISKAKGQPREPQVYTLPPSRDELTKNQVSL | sequence |
| | TCLVKGFYPSDIAVEWESNGQPENNYKTTPPVLDSDGSFFLYSKLTVDKS | |
| | RWQQGNVFSCSVMHEALHNHYTQKSLSLSPGK | |
| 6 | GGGGGIEGPTLRQWLAARAGGGGGGGGIEGPTLRQWLAARAGGGGGGGGI | Functional |
| | EGPTLRQWLAARA | peptide |
| | | fragment with |
| | | three repeats |
| | | of a TPOR |
| | | binding and/or |
| | | activating |
| | | domain |
| 7 | IEGPTLRQWLAARA | TPOR binding |
| | | and/or |
| | | activating |
| | | domain |
| 8 | MAPVAVWAALAVGLELWAAAHALPAQVAFTPYAPEPGSTCRLREYYDQTA | Human TNFR2 |
| | QMCCSKCSPGQHAKVFCTKTSDTVCDSCEDSTYTQLWNWVPECLSCGSRC | isoform 1 |
| | SSDQVETQACTREQNRICTCRPGWYCALSKQEGCRLCAPLRKCRPGFGVA | |
| | RPGTETSDVVCKPCAPGTFSNTTSSTDICRPHQICNVVAIPGNASMDAVC | |
| | TSTSPTRSMAPGAVHLPQPVSTRSQHTQPTPEPSTAPSTSFLLPMGPSPP | |
| | AEGSTGDFALPVGLIVGVTALGLLIIGVVNCVIMTQVKKKPLCLQREAKV | |
| | PHLPADKARGTQGPEQQHLLITAPSSSSSSLESSASALDRRAPTRNQPQA | |
| | PGVEASGAGEARASTGSSDSSPGGHGTQVNVTCIVNVCSSSDHSSQCSSQ | |
| | ASSTMGDTDSSPSESPKDEQVPFSKEECAFRSQLETPETLLGSTEEKPLP | |
| | LGVPDAGMKPS | |
| 9 | GGGGGIEGPTLRQWLAARAGGGGGGGGIEGPTLRQWLAARA | Functional |
| | | peptide |
| | | fragment with |
| | | two repeats of |
| | | a TPOR binding |
| | | and/or |
| | | activating |
| | | domain |
| 10 | LPAQVAFTPYAPEPGSTCRLREYYDQTAQMCCSKCSPGQHAKVFCTKTSD | TNFR2-TPO |
| | TVCDSCEDSTYTQLWNWVPECLSCGSRCSSDQVETQACTREQNRICTCRP | mimetic fusion |
| | GWYCALSKQEGCRLCAPLRKCRPGFGVARPGTETSDVVCKPCAPGTFSNT | polypeptide |
| | TSSTDICRPHQICNVVAIPGNASMDAVCTSTSPTRSMAPGAVHLPQPVST | amino acid |
| | RSQHTQPTPEPSTAPSTSFLLPMGPSPPAEGSTGDEPKSCDKTHTCPPCP | sequence |
| | APELLGGPSVFLFPPKPKDTLMISRTPEVTCVVVDVSHEDPEVKFNWYVD | (without |
| | GVEVHNAKTKPREEQYQSTYRVVSVLTVLHQDWLNGKEYKCKVSNKALPA | signal |
| | PIEKTISKAKGQPREPQVYTLPPSRDELTKNQVSLTCLVKGFYPSDIAVE | peptide) |
| | WESNGQPENNYKTTPPVLDSDGSFFLYSKLTVDKSRWQQGNVFSCSVMHE | |
| | ALHNHYTQKSLSLSPGKGGGGGIEGPTLRQWLAARAGGGGGGGGIEGPTL | |
| | RQWLAARA | |

1. A polypeptide comprising a tumor necrosis factor (TNF) binding and/or inhibiting moiety and a thrombopoietin receptor (TPOR) binding and/or activating moiety.

2. The polypeptide of claim 1, wherein the TNF binding and/or inhibiting moiety is a TNFR and IgG Fc fusion protein that binds to TNF-α or an anti-TNF-α antibody, and wherein the TPOR binding and/or activating moiety comprises a TPOR binding and/or activating domain(s).

3. The polypeptide of claim 2, wherein the TNFR is human TNFR2 (p75) or a functional fragment or variant thereof and the IgG Fc is human IgG Fc.

4. The polypeptide of claim 1, wherein the TNF binding and/or inhibiting moiety is an extracellular portion of human TNFR2 and human Ig Fc fusion protein, or an anti-TNF-α antibody selected from: infliximab (e.g., Remicade®) or a biosimilar, bioequivalent, or biobetter thereof; golimumab (e.g., Simponi®) or a biosimilar, bioequivalent, or biobetter thereof; adalimumab (e.g., Humira®) or a biosimilar, bioequivalent, or biobetter thereof and/or certolizumab pegol (e.g., Cimzia®) or a biosimilar, bioequivalent, or biobetter thereof.

5. (canceled)

6. The polypeptide of claim 1, wherein the TPOR binding and/or activating moiety comprises one, two, three, or more TPOR binding and/or activating domains.

7. The polypeptide of claim 6, comprising one or more spacers between two TPOR binding and/or activating domains.

8. The polypeptide of claim 1, wherein the TPOR binding and/or activating domain is derived from human thrombopoietin (TPO) or comprises a human thrombopoietin mimetic (TPM) peptide.

9. (canceled)

10. (canceled)

11. (canceled)

12. The polypeptide of claim 2, wherein the IgG Fc is a human IgG1, IgG2, IgG3, or IgG4 Fc region or a fragment or variant thereof.

13. (canceled)

14. The polypeptide of claim 1, wherein the C terminus of the TNF binding and/or inhibiting moiety is fused to the N terminus of the TPOR binding and/or activating moiety.

15. The polypeptide of claim 1, comprising a sequence of the formula:
TNFR-Fc-(S₁)ₘ-TPORBD₁-(S₂)ₙ-TPORBD₂-(S₃)ₚ-TPORBD₃,
wherein:
TNFR is a tumor necrosis factor receptor or a fragment or variant thereof;
Fc is an immunoglobulin Fc region or a fragment or variant thereof;
TPORBD₁, TPORBD₂, and TPORBD₃ are the same or different TPOR binding and/or activating domains;
S₁, S₂, and S₃ are the same or different spacers; and
m, n, and p are 0 or greater and are integers independent of one another.

16. The polypeptide of claim 2, wherein the TNFR comprises a sequence having at least 90% to 100% sequence identity with SEQ ID NO: 4.

17. The polypeptide of claim 2, wherein the Fc comprises a sequence having at least 90% to 100% sequence identity with SEQ ID NO: 5.

18. The polypeptide of claim 17, wherein the Fc comprises at least an N-glycosylation site mutation compared to a wild type human IgG Fc, optionally wherein the N-glycosylation site mutation is at N314 according to Kabat numbering (N297 according to EU numbering) which corresponds to N317 in SEQ ID NO: 2.

19. The polypeptide of claim 15, wherein m, n, and p are independently selected from 1 to 10.

20. The polypeptide of claim 15, wherein each of S₁, S₂, and S₃ is a peptide linker.

21. The polypeptide of claim 20, wherein each of S₁, S₂, and S₃ comprises a plurality of glycine, alanine, serine, and/or leucine residues.

22. (canceled)

23. The polypeptide of claim 15, wherein each of TPORBD₁, TPORBD₂, and TPORBD₃ comprises a sequence having at least 80% to 100% sequence identity with SEQ ID NO: 7.

24. The polypeptide of claim 15, wherein (S₁)ₘ-TPORBD₁-(S₂)ₙ-TPORBD₂-(S₃)ₚ-TPORBD₃ comprises a sequence having at least 80% to 100% sequence identity with SEQ ID NO: 6 or SEQ ID NO: 9.

25. The polypeptide of claim 15, wherein the polypeptide comprises a sequence having at least 80% to 100% sequence identity with SEQ ID NO: 2 or SEQ ID NO: 10.

26. The polypeptide of claim 15, further comprising a signal peptide.

27. The polypeptide of claim 26, wherein the signal peptide comprises a sequence having at least 80% to 100% sequence identity with SEQ ID NO: 3.

28. The polypeptide of claim 14, wherein the polypeptide comprises a sequence having at least 80% to 100% sequence identity with SEQ ID NO: 1.

29. (canceled)

30. (canceled)

31. (canceled)

32. (canceled)

33. (canceled)

34. A pharmaceutical composition comprising the polypeptide of claim 1 and a pharmaceutically acceptable carrier or excipient to treat a disease or condition.

35. (canceled)

36. (canceled)

37. (canceled)

38. (canceled)

39. (canceled)

40. (canceled)

41. (canceled)

42. (canceled)

43. A method of producing a secreted polypeptide of claim 1, comprising culturing a eukaryotic host cell comprising the nucleic acid encoding the peptide of claim 1 under conditions suitable for secreting said peptide, and recovering said peptide from the culture medium of the host cell.

44. (canceled)

45. (canceled)

46. A method for treating thrombocytopenia in a subject in need thereof, comprising administering an effective amount of the pharmaceutical composition of claim 34 to the subject.

47. The method of claim 45, wherein the pharmaceutical composition comprises a recombinant fusion protein comprising a sequence having at least 80% to 100% sequence identity with SEQ ID NO: 2 or SEQ ID NO: 10.

48. The method of claim 47, wherein the recombinant fusion protein comprises a dimer of a polypeptide having the sequence set forth in SEQ ID NO: 2 or SEQ ID NO: 1 or SEQ ID NO: 10.

49. (canceled)

50. (canceled)

51. (canceled)

52. The method of claim 46, wherein the thrombocytopenia is caused by and/or associated with an autoimmune disease, liver inflammations and/or damages, drug therapy, radiation therapy, surgery, a chemotherapy, immuno-oncology therapy, and/or combination of chemotherapy and immuno-oncology therapy.

53. The method of claim 46, wherein the thrombocytopenia is caused by and/or associated with liver fibrosis, liver steatosis, hepatitis, non-alcoholic fatty liver disease (NAFLD), immune thrombocytopenia, and/or myelodysplastic syndrome.

54. The method of claim 46, wherein the thrombocytopenia is caused by and/or associated with idiopathic thrombocytopenic purpura (ITP).

55. The method of claim 46, wherein the thrombocytopenia is chemotherapy induced thrombocytopenia (CIT).

56. The method of claim 56, wherein the thrombocytopenia is caused by and/or associated with an immune checkpoint inhibitor therapy.

57. The method of claim 56, wherein immune checkpoint inhibitor therapy treatment with nivolumab, pembrolizumab, dostarlimab, ipilimumab, atezolizumab, avelumab, durvalumab, or cemiplimab, or a biosimilar, bioequivalent, or biobetter thereof, or an antigen binding fragment thereof.

58. The method of claim 46, wherein the peptide is administered subcutaneously, intravenously, intramuscularly, intraperitoneally, or by implantation.

59. The method of claim 46, wherein the peptide is administered in a single dose or a series of doses separated by one or more intervals.

60. The method of claim 46, wherein the peptide is administered weekly, twice a week, once every two weeks, or once every three weeks.

61. (canceled)

62. (canceled)

63. The method of claim 46, wherein the peptide is administered within 24 hours of a first dose of a chemotherapy, immuno-oncology therapy, or combination of chemotherapy and immuno-oncology therapy.

64. The method of claim 46, wherein the peptide is administered at a dose from 0.01 μg/kg to 100 mg/kg based on body weight.

65. (canceled)

66. (canceled)

67. (canceled)

68. The method of claim 43, wherein the eukaryotic host cell is Chinese Hamster cells (CHO).
